(19) United States
(12) Patent Application Publication
(10) Pub. No.: US 2018/0185467 A1
(43) Pub. Date: Jul. 5, 2018

(54) COMPOSITIONS AND METHODS FOR MODULATING VIRAL INFECTION

(71) Applicant: President and Fellows of Harvard College, Cambridge, MA (US)

(72) Inventors: Michaela U. Gack, Brookline, MA (US); Ying K. Chan, Cambridge, MA (US)

(21) Appl. No.: 15/739,428

(22) PCT Filed: Jun. 21, 2016
(86) PCT No.: PCT/US16/38501

§ 371 Date: Dec. 22, 2017

Related U.S. Application Data
(60) Provisional application No. 62/295,635, filed on Feb. 16, 2016.
(60) Provisional application No. 62/183,018, filed on Jun. 22, 2015.

(51) Int. Cl. A61K 39/12; A61K 9/00

(52) U.S. Cl. CPC A61K 39/12; A61K 2039/5254; A61K 9/0019

(57) ABSTRACT

Compositions and methods useful for treating and/or preventing a flavivirus infection are provided.

### RELATED APPLICATIONS

This applications claims the benefit of priority to U.S. Provisional Patent Applications Ser. No. 62/183,018, filed Jun. 22, 2015, and Ser. No. 62/295,635, filed Feb. 16 2016. These applications are hereby incorporated by reference in their entirety.

### BACKGROUND

Flavivirus is a genus of viruses that include West Nile virus, dengue virus, yellow fever virus, Zika virus, and several others. Most of the viruses are transmitted by the bite from an infected arthropod (e.g., mosquito, tick) and cause widespread morbidity and mortality throughout the world. Generally, no specific treatment is available for a flavivirus infection. Current treatments usually involve hospitalization, intravenous fluids, respiratory support, and prevention of secondary infections. Typically, no vaccines against a flavivirus infection exist.

For example, Dengue virus (DV) infects approximately 390 million people annually, and 2.5 billion people live in areas at risk for dengue transmission, making DV the most prevalent arthropod-borne viral pathogen. DV infection can lead to a debilitating febrile disease known as dengue fever, or the more severe and potentially lethal dengue hemorrhagic fever/dengue shock syndrome (DHF/DSS). Four serotypes of DV exist and infection by one serotype only confers long-lasting immunity to that particular serotype. Currently, there are no FDA-approved DV vaccines. Vaccination against only one serotype can lead to DHF/DSS when an individual is subsequently infected with a different serotype due to antibody-dependent enhancement. A tetravalent vaccine candidate recently completed two phase III clinical trials but showed only weak to moderate protection against the widely prevalent DV serotype 2 (DV2) (Capeding et al., Lancet 384, 1358-1365 (2014); Villar et al., N Engl J Med; 372:113-123 (2015)).

Hence, there is a need in the art for broadly effective vaccines and antivirals for flaviviruses.

### SUMMARY

Provided herein are methods and compositions useful in the treatment and/or prevention of a flavivirus infection. In one aspect, provided herein is a mutant flavivirus (e.g., Dengue virus, West Nile virus, Zika virus) comprising a mutated NS3 protein. In some embodiments, the mutated NS3 protein is deficient in 14-3-3ε binding. In some embodiments, a virus comprising the mutated NS3 protein elicits an augmented innate immune response compared to a virus comprising a wild-type NS3 protein. In some embodiments, a virus comprising the mutated NS3 protein produces a stronger inflammatory response in a subject compared to a virus comprising a wild-type NS3 protein. In certain embodiments, a virus comprising the mutant NS3protein induces higher levels of interferon, interferon-stimulated genes, and/or proinflammatory cytokines compared to a virus comprising a wild-type NS3 protein. In certain embodiments, a mutated NS3 protein or mutant virus comprising the mutant protein has a reduced ability to inhibit the translocation of RIG-I to mitochondria/mitochondrial-associated membranes, and RIG-I-dependent signaling compared to a wild-type NS3 protein or a wild-type virus. In some embodiments, a virus comprising the mutated NS3protein elicits a stronger adaptive immune response in primary cells compared to a virus comprising a wild-type NS3 protein.

In certain embodiments, the mutated NS3 protein comprises a mutation between amino acid 63 and amino acid 67 relative to the amino acid sequence of SEQ ID NO: 1, SEQ ID NO: 2, or SEQ ID NO:3 . In certain embodiments, the mutation is an amino acid substitution, insertion, deletion, or combination thereof. In certain embodiments, the mutation comprises a substitution of at least one amino acid between amino acid 63 and amino acid 67 relative to the amino acid sequence of SEQ ID NO: 1, SEQ ID NO: 2, or SEQ ID NO:3 with a different amino acid. In certain embodiments, wherein the mutation comprises a substitution of at least one amino acid between amino acid 63 and amino acid 67 relative to the amino acid sequence of SEQ ID NO: 1, SEQ ID NO: 2, or SEQ ID NO:3, with lysine, e.g., substituting the amino acid at position 64 or 66, or both. In certain embodiments, the mutation comprises a substitution of the amino acids at positions 64 through 66 relative to the amino acid sequence of SEQ ID NO: 1, SEQ ID NO: 2, or SEQ ID NO:3 with the amino acid sequence lysine-isoleucine-lysine.

In certain embodiments, the mutant flavivirus is a dengue virus, a West Nile virus, or a Zika virus. In certain embodiments, the mutant flavivirus is a dengue virus serotype 1, a dengue virus serotype 2, a dengue virus serotype 3, or a dengue virus serotype 4, preferably a dengue virus serotype 2.

In another aspect, provided herein is a pharmaceutical composition comprising a mutant virus disclosed herein, and a pharmaceutically acceptable carrier. In certain embodiments, the pharmaceutical composition further comprises an adjuvant.

In yet another aspect, provided herein is a dengue virus vaccine comprising a mutant dengue virus disclosed herein. In certain embodiments, the dengue virus is a live virus. In certain embodiments, the vaccine further comprises an adjuvant.

In still another aspect, provided herein is a mutated NS3 protein comprising a mutation between amino acid 63 and amino acid 67 relative to the amino acid sequence of SEQ ID NO: 1, SEQ ID NO: 2, or SEQ ID NO:3. In certain embodiments, the mutation is an amino acid substitution, insertion, deletion, or combination thereof. In certain embodiments, the mutation is a substitution of at least one amino acid between amino acid 63 and amino acid 67 relative to the amino acid sequence of SEQ ID NO: 1, SEQ ID NO: 2, or SEQ ID NO:3 with a different amino acid. In certain embodiments, wherein the mutation is a substitution of at least one amino acid between amino acid 63 and amino acid 67 relative to the amino acid sequence of SEQ ID NO: 1, SEQ ID NO: 2, or SEQ ID NO:3, with lysine, e.g., substituting the amino acid at position 64 or 66, or both. In certain embodiments, the mutation corresponds to substituting the amino acids at positions 64 through 66 relative to the amino acid sequence of SEQ ID NO: 1, SEQ ID NO: 2, or SEQ ID NO:3 with the amino acid sequence lysine-isoleucine-lysine. In some embodiments, the protein comprises an amino acid sequence of SEQ ID NO: 4, SEQ ID NO:5, or SEQ ID NO:6.

In another aspect, provided herein is a virus comprising a NS3 protein disclosed herein.

In another aspect, provided herein is a nucleic acid encoding a protein described herein. In some embodiments, the nucleic acid has a sequence of SEQ ID NO: 7, SEQ ID NO: 8, SEQ ID NO 9. In some embodiments, provided herein is a virus comprising a nucleic acid disclosed herein. In some embodiments, provided herein is a vector or an expression vector comprising the nucleic acid. In some embodiments, provided herein is a cell (e.g., a host cell) comprising a vector disclosed herein, expression vector, or nucleic acid. In some embodiments, provided herein is a method for producing a protein. In some embodiments, the method includes culturing a cell comprising a nucleic acid disclosed herein under conditions suitable for expression of the protein. In some embodiments, the method includes isolating the protein from the cell(s) or from the medium in which the cell(s) is cultured. In some embodiments, the method further comprises isolating the protein.

In another aspect, provided herein is a method for inducing in a subject an immune response against a flavivirus comprising administering to the subject a composition comprising a mutant viruses disclosed herein.

In another aspect, provided herein is a method for protecting a subject from a flavivirus, comprising administering to the subject a mutant viruses disclosed herein.

In another aspect, provided herein is a method of treating a viral infection, the method comprising administering a mutant virus disclosed herein, a mutant NS3 protein disclosed herein, a pharmaceutical composition disclosed herein, and/or a vaccine disclosed herein, to a subject (e.g., a subject in need thereof). In certain embodiments, the subject is human. In certain embodiments, the subject is exposed to dengue virus. In certain embodiments, the subject is exposed to a mosquito comprising the dengue virus. In certain embodiments, the subject was exposed to dengue virus or a mosquito, within the last 6 month, within the last month, within the last two weeks, within the last week, within the last 72 hours, within the last 48 hours, within the last 24 hours, within the last 12 hours, within the last 6 hours, within the last 4 hours, within the last 2 hours, or within the last hour.

In certain embodiments, the subject does not have, but is at risk of developing a flavivirus infection. In certain embodiments, the subject is traveling to a region where a flavivirus is prevalent. In certain embodiments, the region is located in the United States, Argentina, Australia, Bangladesh, Barbados, Bolivia, Belize, Brazil, Cambodia, Colombia, Costa Rica, Cuba, Dominican Republic, French Polynesia, Guadeloupe, El Salvador, Grenada, Guatemala, Guyana, Haiti, Honduras, India, Indonesia, Jamaica, Laos, Malaysia, Melanesia, Mexico, Micronesia, Nicaragua, Pakistan, Panama, Paraguay, The Philippines, Puerto Rico, Samoa, Western Saudi Arabia, Singapore, Sri Lanka, Suriname, Taiwan, Thailand, Trinidad and Tobago, Venezuela, Vietnam and/or China.

### BRIEF DESCRIPTION OF THE FIGURES

FIG. 1 includes 8 panels (Panels A-H), which show that the NS3 protein of DV interacts with 14-3-3ε. Panel A is the amino acid sequence of 14-3-3ε and specific peptides identified by MS upon affinity purification of FLAG-NS3-Pro (DV2, strain NGC) from HEK293T cells. Peptide coverage was ˜64.3%. Numbers indicate amino acids. Panel B is an image of an immunoblot showing HEK293T cells transfected with c-myc-tagged 14-3-3ε and FLAG-tagged NS3-Pro or NS3-Hel. 48 h later, WCLs were subjected to FLAG-pulldown (FLAG-PD), followed by immunoblot (IB) with anti-c-myc and anti-FLAG. Panel C is an image of an immunoblot showing binding of FLAG-14-3-3ε and GST, GST-NS5, or GST-NS3, assessed in 293T cells by GST-PD and IB with anti-FLAG and anti-GST antibodies. Panel D is an image of an immunoblot showing HEK293T cells transfected with HA-tagged 14-3-3ε or 14-3-3ε together with GST or GST-NS3. WCLs were subjected to GST-PD, followed by IB with anti-HA and anti-GST. Panel E is an image of an immunoblot showing binding of endogenous 14-3-3ε and GST, or GST-NS3 of DV2 (NGC), YFV (strain 17D), or HCV (strain Conl) in transfected 293T cells, assessed by GST-PD and D3 with anti-14-3-3ε antibody. Panel F is an image of an immunoblot showing Huh7 cells mock-infected or infected with DV2 NGC (MOI 1) for 28 h. WCLs subjected to immunoprecipitation (IP) with anti-14-3-3ε antibody, followed by D3 with anti-NS3 and anti-14-3-3ε. Panel G is an image showing Huh7 cells were transfected with FLAG-14-3-3ε and subsequently mock-infected, or infected with DV2 (NGC) at MOI 1 for 24 h. Cells were stained for FLAG (14-3-3ε;), NS3 and NS4A and imaged by confocal microscopy. Nuclei were stained with DAPI. Panel H is an image of an immunoblot showing in vitro binding of recombinant 14-3-3ε and purified GST or GST-NS3 determined by D3 with anti-14-3-3ε antibody.

FIG. 2 includes 3 panels (Panels A-C), which show that 14-3-3ε is critical for controlling DV replication. Panel A is a bar graph showing Huh7 cells were transfected with empty vector or c-myc-tagged 14-3-3ε and subsequently infected with DV2 NGC at the indicated MOIs. 72 h later, cells were stained for intracellular DV prM and analyzed by flow cytometry. The results are expressed as means±SD (n=3). *p<0.05; **p<0.005. Panel B is a bar graph showing Huh7 cells were transfected with vector or c-myc-tagged 14-3-3ε and subsequently infected with the indicated DV serotypes (MOI 0.05), or HSV-1 (MOI 0.2). Infected cells were determined by intracellular prM (DV) or ICP8 (HSV-1) staining at 72 or 24 h.p.i, respectively. The infectivity for each virus was normalized vector-transfected cells. The results are expressed as means±SD (n=3). **p<0.005. Panel C is a bar graph showing K562 cells were transfected with 14-3-3ε targeting or a non-targeting siRNA (si.C) and 48 h later, cells were infected with DV2 NGC (MOI 1). DV prM+ cells were determined by flow cytometry 48 h.p.i.. The results are expressed as means±SD (n=3). *p<0.05. Knockdown of 14-3-3ε was confirmed by immunoblot.

FIG. 3 includes 5 panels (Panels A-E), which show that NS2B/3 inhibits RIG-I activation independent of proteolytic activity. Panel A is a bar graph depicting IFN-β luciferase activity in 293T cells transfected with GST or GST-RIG-I 2CARD together with vector, DV NS2B/3 WT or S135A, or HCV NS3/4A WT or S139A, normalized to constitutive pGK-β-gal. Viral protein expressions were determined by IB. The results are expressed as means±SD (n=3). **p<0.005. n.s.; not significant. Panel B is a bar graph depicting IFN-β9 luciferase activity in 293T cells transfected with GST or GST-RIG-I 2CARD together with vector, or increasing amounts of DV NS2B/3 WT, NS2B/3 S135A, or NS3, normalized to constitutive pGK-β-gal. The results are expressed as means±SD (n=3). *p<0.05; **p<0.005. Panel C is a bar graph depicting 293T cells, that had been transfected with vector, DV NS2B/3 WT, NS2B/3 S135A, or NS3, subsequently infected with SeV (50 HAU/ml) for 18 h. Luciferase and β-gal activities were determined as in Panel A. A representative immunoblot of viral protein expressions is shown. The results are expressed as means±SD (n=3). *p<0.05; **p<0.005. Panel D is an image of an immunoblot showing GST or GST-NS3 was transfected into HEK293T cells. 48 h later, cells were infected with SeV (50 HAU/ml) for 16 h. WCLs were subjected to native PAGE, followed by immunoblot with anti-IRF3 antibody. WCLs were further used for SDS-PAGE, followed by IB with the indicated antibodies. Panel E is an image of an immunoblot showing GST or GST-NS3 transfected into HEK293T cells similar to Panel D, except WCLs were analyzed by IB 22 h after SeV infection with the indicated antibodies. Expression of PP1γ (not an ISG) was also determined by immunoblot.

FIG. 4 includes 5 panels (Panels A-E), which shows that NS3 inhibits binding of RIG-I to 14-3-3ε, preventing the translocation of activated RIG-I to mitochondria/MAMs. Panel A is a an image of an immunoblot showing HEK293T cells were transfected with empty vector or RIG-I-FLAG together with GST or GST-N53. 48 h later, cells were infected with SeV (50 HAU/ml) for 19 h. WCLs were subjected to FLAG-PD, followed by IB with anti-ubiquitin (Ub) and anti-FLAG antibodies. Panel B is an image of an immunoblot showing Huh7 cells were mock infected, or infected with DV2 NGC (MOI 1) or SeV (50 HAU/ml) for 18 h. WCLs were subjected to IP with anti-RIG-I, followed by IB with anti-Ub and anti-RIG-I. Panel C is an image of an immunoblot showing HEK293T cells were transfected with GST or GST-NS3. 48 h later, cells were infected with SeV (50 HAU/ml) for 23 h. WCLs were subjected to IP with anti-RIG-I (left), or anti-TRIM25 (right), followed by D3 with anti-14-3-3ε, anti-TRIM25 and anti-RIG-I antibodies. Panel D is an immunoblot showing Huh7 cells were mock-infected, or infected with DV2 NGC (MOI 1) or SeV (50 HAU/ml) for 18 h. WCLs were subjected to IP with anti-RIG-I, followed by D3 with anti-14-3-3ε, anti-TRIM25 and anti-RIG-I. Panel E is an immunoblot showing Huh7 cells were mock infected, or infected with DV2 NGC (MOI 1) or SeV (50 HAU/ml) for 22 h. WCLs were subjected to cytosol/mitochondria fractionation, followed by D3 with anti-RIG-I, anti-MAVS and anti-GAPDH antibodies. Expression of RIG-I and NS3 was further determined in the WCL.

FIG. 5 includes 7 panels (Panels A-G), which show that NS3 binds to 14-3-3ε using a phosphomimetic RxEP motif. Panel A is an image of an immunoblot showing 293T cells were transfected with the indicated GST-fused NS3 truncation constructs. 48 h later, WCLs were subjected to GST-PD, followed by IB with anti-14-3-3ε and anti-GST antibodies. Panel B are sequence alignments of amino acid sequence of the NS3 region harboring the 14-3-3 binding motif (green) from DV (serotypes 1-4), and WNV, YFV and HCV. Panel C (left) is a ribbon representation of the crystal structure of DV4 NS3 protein. The protease domain is shown, as well as the linker and helicase domain. The RLEP motif (arrow) is illustrated. Panel C (right) is a close up view of the RLEP motif. Panel D is a bar graph showing bioinformatics analysis of 3280 known DV NS3 protein sequences. The most common residue for each position is shown as the consensus sequence (bottom) and is represented in blue in the bar graph. Polymorphisms for each position are represented by different colors. One polymorphism for E⁶⁶ or P⁶⁷ each was identified, as indicated in parenthesis. Panels E and F are immunoblots showing 293T cells transfected with GST, GST-NS3, or the indicated GST-NS3 mutants. 48 h later, WCLs were subjected to GST-PD, followed by IB with anti-14-3-3ε and anti-GST. Panel G is a bar graph showing HEK293T cells transfected with GST, GST-NS3 WT, or GST-NS3_{KIKP}. 48 h later, cells were infected with SeV (50 HAU/ml). 20 h later, cells were harvested for WCLs or subjected to mitochondria fractionation assay, followed by immunoblot analysis.

FIG. 6 includes 5 panels (Panels A-E), which show a recombinant DV virus encoding a NS3_{KIKP} mutant protein is attenuated in replication and elicits an enhanced antiviral immune response. Panel A is a bar graph showing Huh7 or Huh7.5 cells were infected with DV2_{WT} or DV2_{KIKP} at MOI 0.01. 72 h later, cells were harvested for intracellular prM staining and flow cytometry analysis. The results are expressed as means±SD (n=3). **p<0.005. Panel Bis a series of bar graphs showing Huh7 cells were infected with DV2_{WT} (MOI 0.3) or DV2_{KIKP (MOI} 1), resulting in ˜75% infectivity for both as determined by intracellular prM staining (not shown). 48 h later, total RNA was extracted and transcript levels of indicated genes were determined by quantitative real-time PCR. Transcript levels were normalized against GAPDH and shown as fold levels compared to mock infected cells. The results are expressed as means±SD (n=3). *p<0.05; **p<0.005. Panel C is a bar graph showing Huh7 or Huh7.5 cells were infected with DV2_{KIKP} at MOI 1 and harvested 48 h after infection for qRT-PCR as described in Panel B. The results are expressed as means±SD (n=3). **p<0.005. Panel D is an image of an immunoblot showing Huh7 cells were mock infected, or infected with DV2_{WT} or DV2_{KIKP} at MOI 0.8. 20 h later, cells were harvested for WCLs, or subjected to mitochondria fractionation, followed by immunoblot analysis.

FIG. 7 includes 3 panels (Panels A-C), which show that DV_{KIKP} is defective in antagonizing the innate immune response in primary human monocytes. Panel A is a bar graph showing Primary CD14⁺ monocytes were infected with DV2_{WT} or DV2_{KIKP} at MOI 1 for 24 h. RNA transcript levels were determined by qRT-PCR. The results are expressed as means±SD (n=3). *p<0.05; ***p<0.005. Panel B is a bar graph showing primary CD14+ monocytes were infected as in (A). Supernatants were harvested and analyzed by ELISA for IL-6. The results are expressed as means±SD (n=4). **p<0.005. Panel C is a bar graph showing primary PBMCs were infected as in panel A, and subsequently treated with brefeldin A for 6 h. Cells were harvested for intracellular IFN-β and prM staining and analysis by flow cytometry. Results indicate mean fluorescence intensity (MFI) of IFN-β in prM⁺ monocytes. The results are expressed as means±SD (n=3). *p<0.05.

FIG. 8 includes 2 panels (Panels A and B). Panel A is an image of an immunoblot showing NS2B/3 does not cleave RIG-I, TRIM25 or 14-3-3ε. HEK293T cells transfected with TRIM25-FLAG, RIG-I-FLAG, FLAG-14-3-3 ε or HA-STING together with empty vector or HA-tagged NS2B/3. 48 h later, WCLs were subjected to D3 analysis with anti-HA and anti-FLAG antibodies. Panel A is an image of an immunoblot showing WT NS2B/3, but not the NS2B/3S135A mutant or NS3 expressed alone, is catalytically active. HEK293T cells were transfected with HA-tagged NS2B/3, NS2B/3S135A, or NS3, together with STING-HA. WCLs were analyzed by D3 with anti-HA and anti-actin antibodies.

FIG. 9 includes 2 panels (Panels A and B). Panel A is an immunoblot showing RIG-I, but not TRIM25, directly binds to 14-3-3ε. In vitro binding assay was performed by incubating purified TRIM25-FLAG or RIG-I-FLAG with bacterially purified recombinant (r) human 14-3-3ε. Binding was determined by IB with anti-14-3-3ε and anti-FLAG antibodies. Panel B is an immunoblot showing Ectopic expression of DV NS3 inhibits virus-induced relocalization of endogenous RIG-I from the cytosol to mitochondria. HEK293T cells were transfected with GST or GST-NS3. 48 h later, cells were infected with SeV (50 HAU/ml) for 20 h, followed by mitochondria fractionation assay and IB analysis.

FIG. 10 is a schematic representation of the domain structure of NS3 as well as GST-fused NS3 truncation mutants. 64RxEP67 motif and an overview of the results from the 14-3-3ε binding studies are also indicated. Numbers indicate amino acids.

FIG. 11 includes 3 panels (Panels A-C), which show the NS3KIKP mutant protein is impaired in inhibiting the RIG-I-14-3-3ε interaction and RIG-I translocation to mitochondria. Panel A is an image of an immunoblot showing GST, GST-NS3 WT, or GST-NS3KIKP was transfected into HEK293T cells. 48 h later, cells were infected with SeV (50 HAU/ml) for 22 h. ISG (ISG54 or RIG-I) protein expression in the WCLs was determined by D3 with the indicated antibodies. Panel B is an image of an immunoblot showing HEK293T cells were transfected with GST, GST-NS3 WT, or GST-NS3KIKP. 48 h later, cells were infected with SeV (50 HAU/ml) for 20 h. WCLs were subjected to IP with anti-RIG-I antibody, followed by D3 with anti-14-3-3ε and anti-RIG-I antibodies. Panel C is an image of an immunoblot showing HEK293T cells transfected with GST, GST-NS3 WT, or GST-NS3KIKP. 48 h later, cells were infected with SeV (50 HAU/ml). 20 h later, cells were harvested for WCLs or subjected to mitochondria fractionation assay, followed by D3 analysis.

FIG. 12 includes 5 panels (Panels A-E). Panel A is an image of an immunoblot showing the NS2B/3KIKP mutant protein is catalytically active. HEK293T cells were transfected with HA-tagged STING together with empty vector, or HA-tagged NS2B/3, NS2B/3S135A, or NS2B/3KIKP. 48 h later, WCLs were analyzed by IB with anti-HA and anti-actin antibodies. Panels B and C are bar graphs showing replication of DV2_{WT} and DV2_{KIKP} in Vero cells. Vero cells were infected with DV2_{WT} or DV2_{KIKP} at an MOI of 0.02. Cells were harvested for intracellular prM staining at the indicated time points and analyzed by flow cytometry (Panel B). Furthermore, viral titers were determined in the supernatants (Panel C). The results are expressed as means±SD (n=3). *p<0.05. **p<0.005. Panel D is a bar graph showing replication of DV2_{WT} and DV2_{KIKP} in Huh7 and Huh7.5 cells. Huh7 or Huh7.5 cells were infected with DV2_{WT} or DV2_{KIKP} at an MOI of 0.01. Cells were harvested for intracellular prM staining and analyzed by flow cytometry. The results are from 2 independent experiments and expressed as means±SD (n=6). (E) DV2_{KIKP}, but not DV_{WT}, strongly induces ISG protein expression. A549 cells were infected with DV2_{WT} or DV2_{KIKP} (both MOI 0.2) for 24 h and subjected to immunofluorescence staining of endogenous ISG54 or RIG-I, NS3 and DAPI .

FIG. 13 includes two panels (Panels A and B). Panel A is a bar graph showing increased STAT1 activation in T cells co-cultured with DV2_{KIKP}-infected moDCs. Primary moDCs were infected with DV2WT or DV2KIKP at MOI 1 and co-cultured with syngeneic naive pan T cells at 1:1 ratio. 72 hours later, T cells were harvested and analyzed for intracellular phosphorylated STAT1 (pY701) staining. The results are expressed as means±SD (n=3). *p<0.05. Panel B is a bar graph showing enhanced IFN-γ secretion in T cells co-cultured with DV2_{KIKP}-infected moDCs. Primary moDCs were infected with DV2_{WT} or DV2_{KIKP} at MOI 1 and co-cultured with syngeneic naive pan T cells at 1:1 ratio. 96 hours later, supernatants were harvested and analyzed by ELISA for IFN-γ. The results are expressed as means±SD (n=3 per donor). *p<0.05.

FIG. 14 shows the amino acid sequence of the NS3 region harboring the 14-3-3-binding motif from DV (serotypes 1-4), West Nile Virus (WNV), Yellow Fever Virus (YFV) and HCV.

FIG. 15 is an immunoblot showing binding of GST, GST_NS3 of DV (DV2 NGC), YFV (17D), or WNV (NY99 or kunjin) in transfected HEK293T cells, assessed by GST-PD and IB with anti-14-3-3ε antibody.

FIG. 16 shows IFN-luciferase activity in HEK293T cells transfected with GST-RIG-I 2 CARD together with GST, or GST-NS3 of DV (DV2 NGC) or WNV (NY99 or Kunjin), normalized to constitutive pGK-b-gal. Viral NS3 expressions were determined by immunoblot with an anti-GST antibody. The results are expressed as means±SD (n=3). *p<0.05.

FIG. 17 is an immunoblot showing binding of GST, WNV GST-NS3_{WT} (NY99) or its mutant (GST-NS3_{KIKP}) and endogenous 14-3-3ε in transfected HEK293T cells by GST-PD an IB with an anti-14-3-3ε antibody.

### DETAILED DESCRIPTION

In certain aspects, provided herein are methods and compositions related to the treatment and/or prevention of flavivirus infection, such as DV infection, WNV infection, or a Zika virus (ZV) infection. In some embodiments, disclosed herein are proteins (e.g., variant polypeptides and fragments thereof), nucleic acids encoding the proteins, methods for the production of proteins, and methods for the use of viruses comprising such proteins in various applications, such as methods for treating and/or vaccinating against a number of conditions including, but not limited to, flavivirus infections such as dengue virus. While in no way intended to be limiting, exemplary variant proteins, nucleic acids, and methods for making and using any of the foregoing are described below.

Flavivirus is a genus of viruses that includes, but is not limited to, Absettarov virus, Alkhurma virus (ALKV), Deer tick virus (DT), Gadgets Gully virus (GGYV), Kadam virus (KADV), Karshi virus, Kyasanur Forest disease virus (KFDV), Langat virus (LGTV), Louping ill virus (LIV), Mogiana tick virus (MGTV), Ngoye virus (NGOV), Omsk hemorrhagic fever virus (OHFV), Powassan virus (POWV), Royal Farm virus (RFV), Sokuluk virus (SOKV), Tick-borne encephalitis virus (TBEV), Turkish sheep encephalitis virus (TSE), Kama virus (KAMV), Meaban virus (MEAV), Saumarez Reef virus (SREV), Tyuleniy virus (TYUV), Aedes flavivirus, Barkedji virus, Calbertado virus, Cell fusing agent virus, Chaoyang virus, Culex flavivirus, Culex theileri flavivirus, Culiseta flavivirus, Donggang virus, Hanko virus, Ilomantsi virus, Kamiti River virus, Lammi virus, Marisma mosquito virus, Nakiwogo virus, Nounané virus, Nhumirim virus, Nienokoue virus, Palm Creek virus (PCV), Spanish Culex flavivirus, Spanish Ochlerotatus flavivirus, Quang Binh virus, Aroa virus (AROAV), Bussuquara virus (BSQV), Iguape virus (IGUV), Dengue virus (DENV), Kedougou virus (KEDV), Bussuquara virus, Cacipacore virus (CPCV), Koutango virus (KOUV), Ilheus virus (ILHV), Japanese encephalitis virus (JEV), Murray Valley encephalitis virus (MVEV), Alfuy virus, Rocio virus (ROCV), St. Louis encephalitis virus (SLEV), Usutu virus (USUV), West Nile virus (WNV), Yaounde virus (YAOV), Kokobera virus (KOKV), New Mapoon virus (NMV), Stratford virus (STRV), Bagaza virus (BAGV), Baiyangdian virus (BYDV), Duck egg drop syndrome virus (BYDV), Ilheus virus (ILHV), Jiangsu virus (JSV), Israel turkey meningoencephalomyelitis virus (ITV), Ntaya virus (NTAV), Tembusu virus (TMUV), Spondweni virus (SPOV), Zika virus (ZIKV), Banzi virus (BANV), Bouboui virus (BOUV), Edge Hill virus (EHV), Jugra virus (JUGV), Saboya virus (SABV), Sepik virus (SEPV), Uganda S virus (UGSV), Wesselsbron virus (WESSV), Yellow fever virus (YFV), Tamana bat virus (TABV), Entebbe bat virus (ENTV), Sokoluk virus, Yokose virus (YOKV), Apoi virus (APOIV), Cowbone Ridge virus (CRV), Jutiapa virus (JUTV), Modoc virus (MODV), Sal Vieja virus (SVV), San Perlita virus (SPV), Bukalasa bat virus (BBV), Carey Island virus (CIV), Dakar bat virus (DBV), Montana myotis leukoencephalitis virus (MMLV), Phnom Penh bat virus (PPBV), Rio Bravo virus (RBV), Soybean cyst nematode virus 5, Aedes flavivirus, Aedes cinereus flavivirus, Aedes vexans flavivirus, Culex theileri flavivirus.

As disclosed herein, the NS3 protein of DV antagonizes the RIG-I (retinoic acid-inducible gene-I)-mediated IFN response through a proteolysis-independent mechanism. While the disclosure is not limited by any particular theory or mechanism of action, NS3 binds to the trafficking molecule 14-3-3ε, blocking the translocation of RIG-I to mitochondria/MAMs and thereby inhibiting antiviral signal transduction. NS3 binds to 14-3-3ε using a highly conserved four-amino-acid sequence that mimics a canonical phospho-serine/threonine (pS/pT) motif found in cellular interaction partners of 14-3-3 proteins. Thus, a recombinant DV encoding a mutant NS3 protein deficient in 14-3-3ε binding reduces it's the ability to antagonize RIG-I and elicits an augmented innate immune response.

Thus, in certain embodiments, disclosed herein is a mutant dengue virus comprising a mutated NS3 protein, wherein the mutated NS3 protein is deficient in 14-3-3ε binding. In some embodiments, a mutated NS3 protein produces a stronger inflammatory response in a subject. In certain embodiments, a mutated NS3 protein or mutant virus comprising the mutant protein induces higher levels of interferon, interferon -stimulated genes, and proinflammatory cytokines. In certain embodiments, a mutated NS3 protein or mutant virus comprising the mutant protein has a reduced ability to inhibit the translocation of RIG-I to mitochondria/mitochondrial-associated membranes, and RIG-I-dependent signaling.

### Mutant NS3 Proteins

In certain aspects, provided herein are mutant flavivirus NS3 proteins. In some embodiments, the protein is a variant of the NS3 protein expressed by a dengue virus. An exemplary amino acid sequence for a wild type NS3 protein from dengue virus serotype 2 is as follows (SEQ ID NO: 1):

| | |
|---|---|
| 1 | AGVLWDVPSP PPMGKAELED GAYRIKQKGI LGYSQIGAGV YKEGTFHTMW HVTRGAVLMH |
| 61 | KGKRIEPSWA DVKKDLISYG GGWKLEGEWK EGEEVQVLAL EPGKNPRAVQ TKPGLFKTNA |
| 121 | GTIGAVSLDF SPGTSGSPII DKKGKVVGLY GNGVVTRSGA YVSAIAQTEK SIEDNPEIED |
| 181 | DIFRKRRLTI MDLHPGAGKT KRYLPAIVRE AIKRGLRTLI LAPTRVVAAE MEEALRGLPI |
| 241 | RYQTPAIRAE HTGREIVDLM CHATFTMRLL SPVRVPNYNL IIMDEAHFTD PASIAARGYI |
| 301 | STRVEMGEAA GIFMTATPPG SRDPFPQSNA PIIDEEREIP ERSWNSGHEW VTDFKGKTVW |
| 361 | FVPSIKAGND IAACLSKNGK KVIQLSRKTF DSEYAKTRTN DWDFVVTTDI SEMGANFKAE |
| 421 | RVIDPRRCMK PVILTDGEER VILAGPMPVT HSSAAQRRGR IGRNPKNEND QYIYMGEPLE |
| 481 | NDEDCAHWKE AKMLLDNINT PEGIIPSMFE PEREKVDAID GEYRLRGEAR TTFVDLMRRG |
| 541 | DLPVWLAYRV AAEGINYADR RWCFDGVKNN QILEENVEVE IWTKEGERKK LKPRWLDARI |
| 601 | YSDPLALKEF KEFAAGRK |

In some embodiments, the protein is a variant of the NS3 protein expressed by a West Nile virus. An exemplary amino acid sequence for a wild type NS3 protein for a West Nile Virus is as follows (SEQ ID NO: 2):

| | |
|---|---|
| 1 | GGVLWDTPSP KEYKKGDTTT GVYRIMTRGL LGSYQAGAGV MVEGVFHTLW HTTKGAALMS |
| 61 | GEGRLDPYWG SVKEDRLCYG GPWKLQHKWN GQDEVQMIVV EPGKNVKNVQ TKPGVFKTPE |
| 121 | GEIGAVTLDF PTGTSGSPIV DKNGDVIGLY GNGVIMPNGS YISAIVQGER MDEPIPAGFE |
| 181 | PEMLRKKQIT VLDLHPGAGK TRRILPQIIK EAINRRLRTA VLAPTRVVAA EMAEALRGLP |
| 241 | IRYQTSAVPR EHNGNEIVDV MCHATLTHRL MSPHRVPNYN LEVMDEAHFT DPASIAARGY |
| 301 | ISTKVELGEA AAIFMTATPP GTSDPFPESN SPISDLQTEI PDRAWNSGYE WITEYTGKTV |
| 361 | WFVPSVKMGN EIALCLQCAG KKVVQLNRKS YETEYPKCKN DDWDFVITTD ISEMGANFKA |
| 421 | SRVIDSRKSV KPTIITEGEG RVILGEPSAV TAASAAQRRG RIGRNPSQVG DEYCYGGHTN |
| 481 | EDDSNFAHWT EARIMLDNIN MPNGLIAQFY QPEREKVYTM DGEYRLRGEE RKNFLELLRT |
| 541 | ADLPVWLAYK VAAAGVSYHD RRWCFDGPRT NTILEDNNEV EVITKLGERK TLRPRWIDAR |
| 601 | VYSDHQALKA FKDFASGKR |

In some embodiments, the protein is a variant of the NS3 protein expressed by a Zika virus. An exemplary amino acid sequence for a wild type NS3 protein for a Zika Virus is as follows (SEQ ID NO: 3):

| | |
|---|---|
| 1 | SGALWDVPAP KEVKKGETTD GVYRVMTRRL LGSTQVGVGV MQEGVFHTMW HVTKGAALRS |
| 61 | GEGRLDPYWG DVKQDLVSYC GPWKLDAAWD GLSEVQLLAV PPGERARNIQ TLPGIFKTKD |
| 121 | GDIGAVALDY PAGTSGSPIL DKCGRVIGLY GNGVVIKNGS YVSAITQGKR EEETPVECFE |
| 181 | PSMLKKKQLT VLDLHPGAGK TRRVLPEIVR EAIKKRLRTV ILAPTRVVAA EMEEALRGLP |
| 241 | VRYMTTAVNV THSGTEIVDL MCHATFTSRL LQPIRVPNYN LNIMDEAHFT DPSSIAARGY |
| 301 | ISTRVEMGEA AAIFMTATPP GTRDAFPDSN SPIMDTEVEV PERAWSSGFD WVTDHSGKTV |
| 361 | WFVPSVRNGN EIAACLTKAG KRVIQLSRKT FETEFQKTKN QEWDFVITTD ISEMGANFKA |
| 421 | DRVIDSRRCL KPVILDGERV ILAGPMPVTH ASAAQRRGRI GRNPNKPGDE YMYGGGCAET |
| 481 | DEGHAHWLEA RMLLDNIYLQ DGLIASLYRP EADKVAAIEG EFKLRTEQRK TFVELMKRGD |
| 541 | LPVWLAYQVA SAGITYTDRR WCFDGTTNNT IMEDSVPAEV WTKYGEKRVL KPRWMDARVC |
| 601 | SDHAALKSFK EFAAGKR |

In some embodiments, the variant NS3 protein is deficient in 14-3-3ε binding. In some embodiments, a virus comprising the variant NS3 protein produces a stronger inflammatory response in a subject than a wild-type virus. In certain embodiments, a virus comprising the variant NS3 protein induces higher levels of interferon, interferon—stimulated genes, and/or proinflammatory cytokines than a wild-type virus. In certain embodiments, a variant NS3 protein has a reduced ability to inhibit the translocation of RIG-I to mitochondril/mitochondrial-associated membranes, and RIG-I-dependent signaling than wild-type NS3 protein. In some embodiments, a virus comprising the variant NS3protein elicits a stronger adaptive immune response in primary cells than a wild-type virus.

The variant proteins described herein comprise one or more amino acid substitutions, insertions, or deletions, relative to the wild-type NS3 protein from which they were derived. In some embodiments, a variant protein comprises at least one (e.g., at least two, three, four, five, six, seven, eight, nine, 10, 15, 20, 25, 30, 35, 40, 45, 50, 55, 60, 65, 70, 75, 80, 85, 90, 95, 100, or more than 100) amino acid substitutions, deletions, or insertions, relative to the wild-type, full-length NS3 protein from which it was derived. In some embodiments, a variant protein comprises no more than 150 (e.g., no more than 145, 140, 135, 130, 125, 120, 115, 110, 105, 100, 95, 90, 85, 80, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, 5, 4, 3, 2, or 1) amino acid substitution(s), deletion(s), or insertion(s), relative to the wild-type, full-length NS3 protein from which it was derived.

“Polypeptide,” “peptide,” and “protein” are used interchangeably and mean any peptide-linked chain of amino acids, regardless of length or post-translational modification.

In some embodiments, a variant protein described herein, or a fragment thereof, includes an amino acid substitution between amino acid position 30 and amino acid position 90 relative to SEQ ID NO:1, SEQ ID NO:2, or SEQ ID NO:3; an amino acid substitution between amino acid position 40 and amino acid position 80 relative to SEQ ID NO:1, SEQ ID NO:2, or SEQ ID NO:3; an amino acid substitution between amino acid position 50 and amino acid position 80 relative to SEQ ID NO:1, SEQ ID NO:2, or SEQ ID NO:3; an amino acid substitution between amino acid position 50 and amino acid position 75 relative to SEQ ID NO:1, SEQ ID NO:2, or SEQ ID NO:3; an amino acid substitution between amino acid position 55 and amino acid position 75 relative to SEQ ID NO:1, SEQ ID NO:2, or SEQ ID NO:3; an amino acid substitution between amino acid position 60 and amino acid position 75 relative to SEQ ID NO:1, SEQ ID NO:2, or SEQ ID NO:3; an amino acid substitution between amino acid position 60 and amino acid position 70 relative to SEQ ID NO:1, SEQ ID NO:2, or SEQ ID NO:3; an amino acid substitution between amino acid position 61 and amino acid position 70 relative to SEQ ID NO:1, SEQ ID NO:2, or SEQ ID NO:3; an amino acid substitution between amino acid position 61 and amino acid position 69 relative to SEQ ID NO:1, SEQ ID NO:2, or SEQ ID NO:3; an amino acid substitution between amino acid position 62 and amino acid position 69 relative to SEQ ID NO:1, SEQ ID NO:2, or SEQ ID NO:3; an amino acid substitution between amino acid position 62 and amino acid position 68 relative to SEQ ID NO:1, SEQ ID NO:2, or SEQ ID NO:3; an amino acid substitution between amino acid position 63 and amino acid position 68 relative to SEQ ID NO:1, SEQ ID NO:2, or SEQ ID NO:3; or an amino acid substitution between amino acid position 63 and amino acid position 67 relative to SEQ ID NO:1, SEQ ID NO:2, or SEQ ID NO:3.

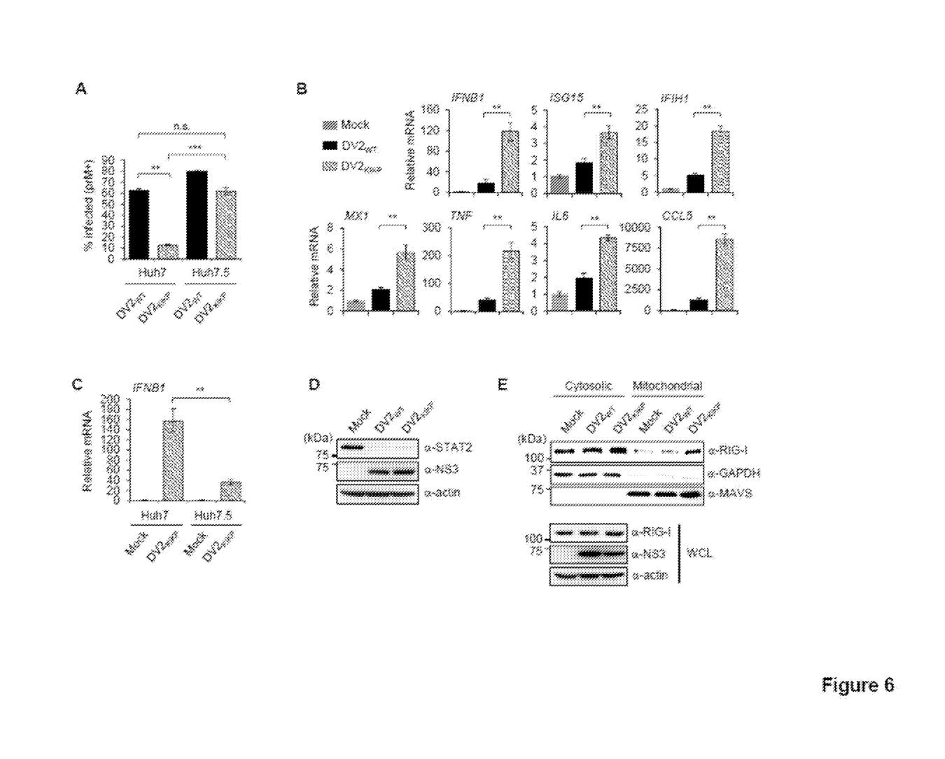

In certain embodiments, a variant protein described herein, includes a substitution of at least one amino acid between amino acid 63 and amino acid 67 relative to the amino acid sequence of SEQ ID NO: 1, SEQ ID NO:2, or SEQ ID NO:3 with a different amino acid e.g., a substitution of an amino acid at position 64 or 66 with lysine. The amino acids at position 64 and 66, relative to SEQ ID NO:1 are two of several amino acids (RxEP) highly conserved among dengue virus NS3 proteins (FIG. 6, Panel B). However, the exact position of these amino acid residues in a given polypeptide varies from species to species and with truncations or extension of the naturally-occurring sequence. In some embodiments, a variant protein includes a substitution of the amino acids at position 64 through 66 with the sequence lysine-isoleucine-lysine.

In some embodiments, the variant protein described herein comprises a substitution at position 64 having the following amino acid sequence (SEQ ID NO: 4):

| | |
|---|---|
| 1 | AGVLWDVPSP PPMGKAELED GAYRIKQKGI LGYSQIGAGV YKEGTFHTMW HVTRGAVLMH |
| 61 | KGKKIEPSWA DVKKDLISYG GGWKLEGEWK EGEEVQVLAL EPGKNPRAVQ TKPGLFKTNA |
| 121 | GTIGAVSLDF SPGTSGSPII DKKGKVVGLY GNGVVTRSGA YVSAIAQTEK SIEDNPEIED |
| 181 | DIFRKRRLTI MDLHPGAGKT KRYLPAIVRE AIKRGLRTLI LAPTRVVAAE MEEALRGLPI |
| 241 | RYQTPAIRAE HTGREIVDLM CHATFTMRLL SPVRVPNYNL IIMDEAHFTD PASIAARGYI |
| 301 | STRVEMGEAA GIFMTATPPG SRDPFPQSNA PIIDEEREIP ERSWNSGHEW VTDFKGKTVW |
| 361 | FVPSIKAGND IAACLSKNGK KVIQLSRKTF DSEYAKTRTN DWDFVVTTDI SEMGANFKAE |
| 421 | RVIDPRRCMK PVILTDGEER VILAGPMPVT HSSAAQRRGR IGRNPKNEND QYIYMGEPLE |
| 481 | NDEDCAHWKE AKMLLDNINT PEGIIPSMFE PEREKVDAID GEYRLRGEAR TTFVDLMRRG |
| 541 | DLPVWLAYRV AAEGINYADR RWCFDGVKNN QILEENVEVE IWTKEGERKK LKPRWLDARI |
| 601 | YSDPLALKEF KEFAAGRK |

In some embodiments, the variant protein described herein comprises a substitution at position 66 having the following amino acid sequence (SEQ ID NO: 5):

| | |
|---|---|
| 1 | AGVLWDVPSP PPMGKAELED GAYRIKQKGI LGYSQIGAGV YKEGTFHTMW HVTRGAVLMH |
| 61 | KGKRIKPSWA DVKKDLISYG GGWKLEGEWK EGEEVQVLAL EPGKNPRAVQ TKPGLFKTNA |
| 121 | GTIGAVSLDF SPGTSGSPII DKKGKVVGLY GNGVVTRSGA YVSAIAQTEK SIEDNPEIED |
| 181 | DIFRKRRLTI MDLHPGAGKT KRYLPAIVRE AIKRGLRTLI LAPTRVVAAE MEEALRGLPI |
| 241 | RYQTPAIRAE HTGREIVDLM CHATFTMRLL SPVRVPNYNL IIMDEAHFTD PASIAARGYI |
| 301 | STRVEMGEAA GIFMTATPPG SRDPFPQSNA PIIDEEREIP ERSWNSGHEW VTDFKGKTVW |
| 361 | FVPSIKAGND IAACLSKNGK KVIQLSRKTF DSEYAKTRTN DWDFVVTTDI SEMGANFKAE |
| 421 | RVIDPRRCMK PVILTDGEER VILAGPMPVT HSSAAQRRGR IGRNPKNEND QYIYMGEPLE |
| 481 | NDEDCAHWKE AKMLLDNINT PEGIIPSMFE PEREKVDAID GEYRLRGEAR TTFVDLMRRG |
| 541 | DLPVWLAYRV AAEGINYADR RWCFDGVKNN QILEENVEVE IWTKEGERKK LKPRWLDARI |
| 601 | YSDPLALKEF KEFAAGRK |

In some embodiments, the variant protein described herein comprises a substitution at position 64and position 66 having the following amino acid sequence (SEQ ID NO: 6):

| | |
|---|---|
| 1 | AGVLWDVPSP PPMGKAELED GAYRIKQKGI LGYSQIGAGV YKEGTFHTMW HVTRGAVLMH |
| 61 | KGKKIKPSWA DVKKDLISYG GGWKLEGEWK EGEEVQVLAL EPGKNPRAVQ TKPGLFKTNA |
| 121 | GTIGAVSLDF SPGTSGSPII DKKGKVVGLY GNGVVTRSGA YVSAIAQTEK SIEDNPEIED |
| 181 | DIFRKRRLTI MDLHPGAGKT KRYLPAIVRE AIKRGLRTLI LAPTRVVAAE MEEALRGLPI |
| 241 | RYQTPAIRAE HTGREIVDLM CHATFTMRLL SPVRVPNYNL IIMDEAHFTD PASIAARGYI |
| 301 | STRVEMGEAA GIFMTATPPG SRDPFPQSNA PIIDEEREIP ERSWNSGHEW VTDFKGKTVW |
| 361 | FVPSIKAGND IAACLSKNGK KVIQLSRKTF DSEYAKTRTN DWDFVVTTDI SEMGANFKAE |
| 421 | RVIDPRRCMK PVILTDGEER VILAGPMPVT HSSAAQRRGR IGRNPKNEND QYIYMGEPLE |
| 481 | NDEDCAHWKE AKMLLDNINT PEGIIPSMFE PEREKVDAID GEYRLRGEAR TTFVDLMRRG |
| 541 | DLPVWLAYRV AAEGINYADR RWCFDGVKNN QILEENVEVE IWTKEGERKK LKPRWLDARI |
| 601 | YSDPLALKEF KEFAAGRK |

As used herein, the term “conservative substitution” refers to the replacement of an amino acid present in the native sequence in a given polypeptide with a naturally or non-naturally occurring amino acid having similar steric properties. Where the side-chain of the native amino acid to be replaced is either polar or hydrophobic, the conservative substitution should be with a naturally occurring amino acid, a non-naturally occurring amino acid that is also polar or hydrophobic, and, optionally, with the same or similar steric properties as the side-chain of the replaced amino acid. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. One letter amino acid abbreviations are as follows: alanine (A); arginine (R); asparagine (N); aspartic acid (D); cysteine (C); glycine (G); glutamine (Q); glutamic acid (E); histidine (H); isoleucine (I); leucine (L); lysine (K); methionine (M); phenylalanine (F); proline (P); serine (S); threonine (T); tryptophan (W), tyrosine (Y); and valine (V).

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted.

In some embodiments, a variant protein described herein, or a fragment thereof, has an amino acid sequence that is at least 80 (e.g., at least 81, 82, 83, 84, 85, 86, 87, 88, 89, 90, 91, 92, 93, 94, 95, 96, 97, 98, or 99) % identical to: (i) the amino acid of SEQ ID NO:2; (ii) the amino acid of SEQ ID NO:3; or (iii) the amino acid of SEQ ID NO:4with the proviso that the variant protein or fragment thereof comprises an amino acid substitution at position 64, an amino acid substitution at position 66, or combinations thereof.

In some embodiments, a variant protein described herein, or a fragment thereof, has an amino acid sequence that is at least 80 (e.g., at least 81, 82, 83, 84, 85, 86, 87, 88, 89, 90, 91, 92, 93, 94, 95, 96, 97, 98, or 99) % identical to: (i) the amino acid of SEQ ID NO:4; (ii) the amino acid of SEQ ID NO:5; or (iii) the amino acid of SEQ ID NO:6.

Percent amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software, such as BLAST software or ClustalW2. Appropriate parameters for measuring alignment, including any algorithms needed to achieve maximal alignment over the full-length of the sequences being compared can be determined by known methods.

The proteins disclosed herein (e.g., a mutant NS3 proteins) can be produced using any appropriate technique in the art. For example, a nucleic acid encoding a fusion protein can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Several possible vector systems are available for the expression of recombinant proteins from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed, or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), cytomegalovirus, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, viral infection, dextran-mediated transfection, polybrene-mediated transfection, protoplast fusion, and direct microinjection.

Appropriate host cells for the expression of recombinant proteins include yeast, bacteria, insect, plant, and mammalian cells (e.g., rodent cell lines, such as Chinese Hamster Ovary (CHO) cells). Of particular interest are bacteria such as E. coli, fungi such as Saccharomyces cerevisiae and Pichia pastoris, insect cells such as SF9, mammalian cell lines (e.g., human cell lines), as well as primary cell lines.

A protein can be produced from the cells by culturing a host cell transformed with the expression vector containing nucleic acid encoding the polypeptide, under conditions, and for an amount of time, sufficient to allow expression of the proteins. Such conditions for protein expression will vary with the choice of the expression vector and the host cell, and will be easily ascertained by one skilled in the art through routine experimentation. For example, proteins expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001)). The choice of codons, suitable expression vectors and suitable host cells will vary depending on a number of factors, and may be easily optimized as needed. A fusion protein described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the recombinant proteins can be isolated. The term “purified” or “isolated” as applied to any of the proteins described herein refers to a polypeptide that has been separated or purified from components (e.g., proteins or other naturally-occurring biological or organic molecules) which naturally accompany it, e.g., other proteins, lipids, and nucleic acid in a prokaryotic or eukaryotic cell expressing the proteins. Typically, a polypeptide is purified when it constitutes at least 60 (e.g., at least 65, 70, 75, 80, 85, 90, 92, 95, 97, or 99) %, by weight, of the total protein in a sample.

The recombinant proteins can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) “Protein Purification, 3^{rd} edition,” Springer-Verlag, New York City, N.Y. The degree of purification necessary will vary depending on the desired use. In some instances, no purification of the expressed proteins will be necessary.

Methods for determining the yield or purity of a purified protein are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

The expression of a protein (e.g., a mutant NS3 protein disclosed herein) can also be determined by detecting and/or measuring expression of a protein. Methods of determining protein expression generally involve the use of antibodies specific for the target protein of interest. For example, methods of determining protein expression include, but are not limited to, western blot or dot blot analysis, immunohistochemistry (e.g., quantitative immunohistochemistry), immunocytochemistry, enzyme-linked immunosorbent assay (ELISA), enzyme-linked immunosorbent spot (ELISPOT; Coligan et al., eds. (1995) Current Protocols in Immunology. Wiley, New York), or antibody array analysis (see, e.g., U.S. Patent Application Publication Nos. 20030013208 and 2004171068, the disclosures of each of which are incorporated herein by reference in their entirety). Further description of many of the methods above and additional methods for detecting protein expression can be found in, e.g., Sambrook et al. (supra).

In one example, the presence or amount of protein expression can be determined using a western blotting technique. For example, a lysate can be prepared from a biological sample, or the biological sample itself, can be contacted with Laemmli buffer and subjected to sodium-dodecyl sulfate polyacrylamide gel electrophoresis (SDS-PAGE). SDS-PAGE-resolved proteins, separated by size, can then be transferred to a filter membrane (e.g., nitrocellulose) and subjected to immunoblotting techniques using a detectably-labeled antibody specific to the protein of interest. The presence or amount of bound detectably-labeled antibody indicates the presence or amount of protein in the biological sample.

In another example, an immunoassay can be used for detecting and/or measuring the protein expression of a protein. As above, for the purposes of detection, an immunoassay can be performed with an antibody that bears a detection moiety (e.g., a fluorescent agent or enzyme). Proteins from a biological sample can be conjugated directly to a solid-phase matrix (e.g., a multi-well assay plate, nitrocellulose, agarose, sepharose, encoded particles, or magnetic beads) or it can be conjugated to a first member of a specific binding pair (e.g., biotin or streptavidin) that attaches to a solid-phase matrix upon binding to a second member of the specific binding pair (e.g., streptavidin or biotin). Such attachment to a solid-phase matrix allows the proteins to be purified away from other interfering or irrelevant components of the biological sample prior to contact with the detection antibody and also allows for subsequent washing of unbound antibody. Here as above, the presence or amount of bound detectably-labeled antibody indicates the presence or amount of protein in the biological sample.

Methods for generating antibodies or antibody fragments specific for a protein can be generated by immunization, e.g., using an animal, or by in vitro methods such as phage display. A polypeptide that includes all or part of a target protein can be used to generate an antibody or antibody fragment. The antibody can be a monoclonal antibody or a preparation of polyclonal antibodies.

Methods for detecting or measuring gene expression can optionally be performed in formats that allow for rapid preparation, processing, and analysis of multiple samples. This can be, for example, in multi-welled assay plates (e.g., 96 wells or 386 wells) or arrays (e.g., nucleic acid chips or protein chips). Stock solutions for various reagents can be provided manually or robotically, and subsequent sample preparation (e.g., RT-PCR, labeling, or cell fixation), pipetting, diluting, mixing, distribution, washing, incubating (e.g., hybridization), sample readout, data collection (optical data) and/or analysis (computer aided image analysis) can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay. Exemplary high-throughput cell-based assays (e.g., detecting the presence or level of a target protein in a cell) can utilize ArrayScan® VTI HCS Reader or KineticScan® HCS Reader technology (Cellomics Inc., Pittsburg, Pa.). Exemplary methods for producing, expressing, and isolating a NS3 protein are exemplified in the working examples.

### Mutant Viruses

In certain aspects, provided herein are a recombinant virus comprising a mutant NS3 protein described herein. In certain embodiments, disclosed herein are cDNA of a dengue virus comprising a nucleic acid sequence mutations that encode the mutant NS3 protein.

Suitable cell lines for propagating a recombinant dengue virus include mammalian cells, such as Vero cells, AGMK cells, BHK-21cells, COS-I or COS-7 cells, MDCK cells, CV-I cells, LLC-MK2 cells, primary cell lines such as fetal Rhesus lung (FRhL-2) cells, BSC-I cells, and MRC-5 cells, or human diploid fibroblasts, as well as avian cells, chicken or duck embryo derived cell lines, e.g., AGE1 cells, and primary, chicken embryo fibroblasts, and mosquito cell lines, such as C6/36. To propagate virus in cell culture, a recombinant dengue virus is used to infect the host cell (for example, selected from among the suitable cell types listed above). After virus adsorption, the cultures are fed with medium capable of supporting growth of the cells. The host cells are maintained in culture until the desired virus titer is achieved.

To recover virus, the virus is harvested by common methods known in the art including slow-speed centrifugation or by filtration through a filter of pore size of 0.45 μm. Methods for concentrating recovered virus are within the scope of a person with ordinary skill in the art and include, for example, ultrafiltration (e.g., with a membrane of no greater than 300 kDa pore size), or precipitation with polyethylene glycol (PEG) 8000. Methods for purifying viruses are known to a person with ordinary skill in the art and include continuous or multi-step sucrose gradients, purification by column chromatography using size exclusion, ion exchange, adsorption, or affinity columns, or purification by partitioning in polymer two-phase or multi-phase systems, and any combination thereof. Methods for assaying for virus positive fractions include plaque assay, hemagglutination (HA) assay, and/or antigen assays such as immunoassays.

### Mutant Nucleic Acid Molecules

Provided herein are nucleic acid molecules that encode the mutant NS3 protein described herein. The nucleic acids may be present, for example, in whole cells, in a cell lysate, or in a partially purified or substantially pure form.

In some embodiments, the nucleic acid has a sequence of SEQ ID NO: 7:

gcgggcgtgctgtgggatgtgccgagcccgccgccgatgggcaaagcgga
actggaagatggcgcgtatcgcattaaacagaaaggcattctgggctata
gccagattggcgcgggcgtgtataaagaaggcacctttcataccatgtgg
catgtgacccgcggcgcggtgctgatgcataaaggcaaaaaaattgaacc
gagctgggcggatgtgaaaaaagatctgattagctatggcggcggctgga
aactggaaggcgaatggaaagaaggcgaagaagtgcaggtgctggcgctg
gaaccgggcaaaaacccgcgcgcggtgcagaccaaaccgggcctgtttaa
aaccaacgcggcaccattggcgcggtgagcctggattttagcccgggcac
cageggcagcccgattattgataaaaaaggcaaagtggtgggcctgtatg
gcaacggcgtggtgacccgcagcggcgcgtatgtgagcgcgattgcgcag
accgaaaaaagcattgaagataacccggaaattgaagatgatatttttcg
caaacgccgcctgaccattatggatctgcatccgggcgcgggcaaaacca
aacgctatctgccggcgattgtgcgcgaagcgattaaacgcggcctgcgc
accctgattctggcgccgacccgcgtggtggcggcggaaatggaagaagc
gctgcgcggcctgccgattcgctatcagaccccggcgattcgcgcggaac
ataccggccgcgaaattgtggatctgatgtgccatgcgacctttaccatg
cgcctgctgagcccggtgcgcgtgccgaactataacctgattattatgga
tgaagcgcattttaccgatccggcgagcattgeggcgcgcggctatatta
gcacccgcgtggaaatgggcgaagcggcgggcatttttatgaccgcgacc
ccgccgggcagccgcgatccgtttccgcagagcaacgcgccgattattga
tgaagaacgcgaaattccggaacgcagctggaacageggccatgaatggg
tgaccgattttaaaggcaaaaccgtgtggtttgtgccgagcattaaagcg
ggcaacgatattgcggcgtgcctgagcaaaaacggcaaaaaagtgattca
gctgagccgcaaaacctttgatagcgaatatgcgaaaacccgcaccaaga
ttgggattttgtggtgaccaccgatattagcgaaatgggcgcgaacttta
aagcggaacgcgtgattgatccgcgccgctgcatgaaaccggtgattctg
accgatggcgaagaacgcgtgattctggcgggcccgatgccggtgaccca
tagcagcgcggcgcagcgccgcggccgcattggccgcaacccgaaaaacg
aaaacgatcagtatatttatatgggcgaaccgctggaaaacgatgaagat
tgcgcgcattggaaagaagcgaaaatgctgctggataacattaacacccc
ggaaggcattattccgagcatgtttgaaccggaacgcgaaaaagtggatg
cgattgatggcgaatatcgcctgcgcggcgaagcgcgcaccacctttgtg
gatctgatgcgccgcggcgatctgccggtgtggctggcgtatcgcgtggc
ggcggaaggcattaactatgcggatcgccgctggtgetttgatggcgtga
aaaacaaccagattctggaagaaaacgtggaagtggaaatttggaccaaa
gaaggcgaacgcaaaaaactgaaaccgcgctggctggatgcgcgcattta
tagcgatccgctggcgctgaaagaatttaaagaatttgcggcgggccgca
aa

In some embodiments, the nucleic acid has a sequence of SEQ ID NO: 8:

gcgggcgtgctgtgggatgtgccgagcccgccgccgatgggcaaagcgga
actggaagatggcgcgtatcgcattaaacagaaaggcattctgggctata
gccagattggcgcgggcgtgtataaagaaggcacctttcataccatgtgg
catgtgacccgcggcgcggtgctgatgcataaaggcaaacgcattaaacc
gagctgggcggatgtgaaaaaagatctgattagctatggcggcggctgga
aactggaaggcgaatggaaagaaggcgaagaagtgcaggtgctggcgctg
gaaccgggcaaaaacccgcgcgcggtgcagaccaaaccgggcctgtttaa
aaccaacgcgggcaccattggcgcggtgagcctggattttagcccgggca
ccageggcagcccgattattgataaaaaaggcaaagtggtgggcctgtat
ggcaacggcgtggtgacccgcagcggcgcgtatgtgagcgcgattgcgca
gaccgaaaaaagcattgaagataacccggaaattgaagatgatatttttc
gcaaacgccgcctgaccattatggatctgcatccgggcgcgggcaaaacc
aaacgctatctgccggcgattgtgcgcgaagcgattaaacgcggcctgcg
caccctgattctggcgccgacccgcgtggtggcggcggaaatggaagaag
cgctgcgcggcctgccgattcgctatcagaccccggcgattcgcgcggaa
cataccggccgcgaaattgtggatctgatgtgccatgcgacctttaccat
gcgcctgctgagcccggtgcgcgtgccgaactataacctgattattatgg
atgaagcgcattttaccgatccggcgagcattgeggcgcgcggctatatt
agcacccgcgtggaaatgggcgaagcggcgggcatttttatgaccgcgac
cccgccgggcagccgcgatccgtttccgcagagcaacgcgccgattattg
atgaagaacgcgaaattccggaacgcagctggaacageggccatgaatgg
gtgaccgattttaaaggcaaaaccgtgtggtttgtgccgagcattaaagc
gggcaacgatattgcggcgtgcctgagcaaaaacggcaaaaaagtgattc
agctgagccgcaaaacctttgatagcgaatatgcgaaaacccgcaccaac
gattgggattttgtggtgaccaccgatattagcgaaatgggcgcgaactt
taaagcggaacgcgtgattgatccgcgccgctgcatgaaaccggtgattc
tgaccgatggcgaagaacgcgtgattctggcgggcccgatgccggtgacc
catagcagcgcggcgcagcgccgcggccgcattggccgcaacccgaaaaa
cgaaaacgatcagtatatttatatgggcgaaccgctggaaaacgatgaag
attgcgcgcattggaaagaagcgaaaatgctgctggataacattaacacc
ccggaaggcattattccgagcatgtttgaaccggaacgcgaaaaagtgga
tgcgattgatggcgaatatcgcctgcgcggcgaagcgcgcaccacctttg
tggatctgatgcgccgcggcgatctgccggtgtggctggcgtatcgcgtg
gcggcggaaggcattaactatgcggatcgccgctggtgetttgatggcgt
gaaaaacaaccagattctggaagaaaacgtggaagtggaaatttggacca
aagaaggcgaacgcaaaaaactgaaaccgcgctggctggatgcgcgcatt
tatagcgatccgctggcgctgaaagaatttaaagaatttgcggcgggccg
caaa

In some embodiments, the nucleic acid has a sequence of SEQ ID NO: 9:

gcgggcgtgctgtgggatgtgccgagcccgccgccgatgggcaaagcgga
actggaagatggcgcgtatcgcattaaacagaaaggcattctgggctata
gccagattggcgcgggcgtgtataaagaaggcacctttcataccatgtgg
catgtgacccgcggcgcggtgctgatgcataaaggcaaaaaaattaaacc
gagctgggcggatgtgaaaaaagatctgattagctatggcggcggctgga
aactggaaggcgaatggaaagaaggcgaagaagtgcaggtgctggcgctg
gaaccgggcaaaaacccgcgcgcggtgcagaccaaaccgggcctgtttaa
aaccaacgcgggcaccattggcgcggtgagcctggattttagcccgggca
ccageggcagcccgattattgataaaaaaggcaaagtggtgggcctgtat
ggcaacggcgtggtgacccgcagcggcgcgtatgtgagcgcgattgcgca
gaccgaaaaaagcattgaagataacccggaaattgaagatgatatttttc
gcaaacgccgcctgaccattatggatctgcatccgggcgcgggcaaaacc
aaacgctatctgccggcgattgtgcgcgaagcgattaaacgcggcctgcg
caccctgattctggcgccgacccgcgtggtggcggcggaaatggaagaag
cgctgcgcggcctgccgattcgctatcagaccccggcgattcgcgcggaa
cataccggccgcgaaattgtggatctgatgtgccatgcgacctttaccat
gcgcctgctgagcccggtgcgcgtgccgaactataacctgattattatgg
atgaagcgcattttaccgatccggcgagcattgeggcgcgcggctatatt
agcacccgcgtggaaatgggcgaagcggcgggcatttttatgaccgcgac
cccgccgggcagccgcgatccgtttccgcagagcaacgcgccgattattg
atgaagaacgcgaaattcggaacgcagctggaacageggccatgaatggg
tgaccgattttaaaggcaaaaccgtgtggtttgtgccgagcattaaagcg
ggcaacgatattgcggcgtgcctgagcaaaaacggcaaaaaagtgattca
gctgagccgcaaaacctttgatagcgaatatgcgaaaacccgcaccaacg
attgggattttgtggtgaccaccgatattagcgaaatgggcgcgaacttt
aaagcggaacgcgtgattgatccgcgccgctgcatgaaaccggtgattct
gaccgatggcgaagaacgcgtgattctggcgggcccgatgccggtgaccc
atagcagcgcggcgcagcgccgcggccgcattggccgcaacccgaaaaac
gaaaacgatcagtatatttatatgggcgaaccgctggaaaacgatgaaga
ttgcgcgcattggaaagaagcgaaaatgctgctggataacattaacaccc
cggaaggcattattccgagcatgtttgaaccggaacgcgaaaaagtggat
gcgattgatggcgaatatcgcctgcgcggcgaagcgcgcaccacctttgt
ggatctgatgcgccgcggcgatctgccggtgtggctggcgtatcgcgtgg
cggcggaaggcattaactatgcggatcgccgctggtgetttgatggcgtg
aaaaacaaccagattctggaagaaaacgtggaagtggaaatttggaccaa
agaaggcgaacgcaaaaaactgaaaccgcgctggctggatgcgcgcattt
atagcgatccgctggcgctgaaagaatttaaagaatttgcggcgggccgc
aaa

Nucleic acid molecules provided herein can be obtained using standard molecular biology techniques. For example, nucleic acid molecules described herein can be cloned using standard PCR techniques or chemically synthesized. For nucleic acids encoding antibodies expressed by hybridomas, cDNAs encoding the light and/or heavy chains of the antibody made by the hybridoma can be obtained by standard PCR amplification or cDNA cloning techniques.

In certain embodiments, provided herein are vectors that contain the isolated nucleic acid molecules described herein. As used herein, the term “vector,” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”).

In certain embodiments, provided herein are cells that contain a nucleic acid described herein (e.g., a nucleic acid encoding an NS3 protein described herein). The cell can be, for example, prokaryotic, eukaryotic, mammalian, avian, murine and/or human. In certain embodiments the cell is a hybridoma. In certain embodiments the nucleic acid provided herein is operably linked to a transcription control element such as a promoter. In some embodiments the cell transcribes the nucleic acid provided herein and thereby expresses a protein described herein. The nucleic acid molecule can be integrated into the genome of the cell or it can be extrachromosomal.

### Pharmaceutical Compositions and Vaccines

In certain aspects, provided herein are pharmaceutical compositions and/or vaccines comprising a mutant dengue virus described herein.

In some embodiments, the pharmaceutical compositions and/or vaccines described herein include a virus comprising a mutant NS3 protein together with one or more excipients and/or adjuvants. In some embodiments the pharmaceutical composition and/or vaccine described herein comprises a mutant flavivirus (e.g., DV, WNV, or ZV) viral genome and/or mutant gene encoding mutant NS3. The pharmaceutical composition and/or vaccine can contain genetic material, such as a heterologous gene insert expressing the mutant protein. In such a case, the mutant NS3 can be expressed in cells of a susceptible species immunized with the vaccine containing mutant DV , WNV, or ZV and/or mutant NS3. Immunity against wild type DV, WNV, or ZV can thereby be conferred in a species and/or tissue normally susceptible to a DV, WNV, or ZV infection.

Accordingly, in some embodiments, the mutant NS3 virus has reduced ability to bind to the trafficking molecule 14-3-3ε and reduced ability to block of the translocation of RIG-I to mitochondria/MAMs. Thus, in some embodiments, a mutant flavivirus (e.g., DV, WNV, or ZV) encoding a mutant NS3 protein described herein deficient in 14-3-3ε binding has reduced ability to antagonize RIG-I and elicits an augmented innate immune response. The present disclosure affords a pharmaceutical composition and/or vaccine to treat and/or prevent flavivirus infections or other disease states related to or caused by flavivirus infections, e.g., dengue fever, yellow fever, Zika fever, microcephaly. In some embodiments, the mutant flavivirus is able to induce an immune response in a subject, which results in the treated subject's immune system to fight a wild type flavivirus. In some embodiments, a pharmaceutical composition and/or vaccine having the mutant flavivirus and/or mutant NS3 is taken by subjects who have been infected by flavivirus to improve an immune response to a wild type flavivirus .

In some embodiments, the pharmaceutical composition and/or vaccine may further comprise an adjuvant that can augment the immune response by increasing delivery of antigen, stimulating cytokine production, and/or stimulating antigen presenting cells. In some embodiments, the adjuvant can be administered concurrently with the pharmaceutical composition and/or vaccine composition disclosed herein, e.g., in the same composition or in separate compositions. For example, an adjuvant can be administered prior or subsequent to the pharmaceutical composition and/or vaccine composition disclosed herein. Such adjuvants include, but are not limited to: aluminum salts, non-toxic bacterial fragments, cholera toxin (and detoxified fractions thereof), chitosan, homologous heat-labile of E. coli (and detoxified fractions thereof), lactide/glycolide homo and copolymers (PLA/GA), polyanhydride e.g. trimellitylimido-L-tyrosine, DEAE-dextran, saponins complexed to membrane protein antigens (immune stimulating complexes—ISCOMS), bacterial products such as lipopolysaccharide (LPS) and muramyl dipeptide, (MDP), liposomes, cochelates, proteinoids, cytokines (interleukins, interferons), genetically engineered live microbial vectors, non-infectious pertussis mutant toxin, neurimidase/galactose oxidase, and attenuated bacterial and viral toxins derived from mutant strains.

In some embodiments, the mutant DV is able to induce an immune response in a subject against one, two, three or all four serotypes of the dengue virus (e.g., dengue virus serotype 1, dengue virus serotype 2, dengue virus serotype 3, or dengue virus serotype 4) . In some embodiments, the pharmaceutical composition and/or vaccine may comprise a combination of mutant proteins from two, three or all four serotypes of the dengue virus. In some embodiments, the mutant DV is a dengue virus serotype 2.

In certain embodiments, the pharmaceutical composition , vaccine and/or adjuvant can be administered to a subject, e.g., a human subject, using a variety of methods that depend, in part, on the route of administration. The route can be, e.g., intravenous injection or infusion (IV), subcutaneous injection (SC), intraperitoneal (IP) injection, or intramuscular injection (IM).

### Therapeutic Methods

In certain aspects, provided herein is a method for inducing an immune response against a flavivirus in a subject comprising administering to the subject a composition (e.g., a vaccine composition) disclosed herein. In some embodiments, provided herein is a method for protecting a subject from a flavivirus, comprising administering to the a composition disclosed herein. In some embodiments, provided herein is a method of treating a subject for flavivirus infection comprising administering to the subject a composition disclosed herein.

A “subject,” as used herein, can be any mammal. For example, a subject can be a human, a non-human primate (e.g., monkey, baboon, or chimpanzee), a horse, a cow, a pig, a sheep, a goat, a dog, a cat, a rabbit, a guinea pig, a gerbil, a hamster, a rat, or a mouse. In some embodiments, the subject is an infant (e.g., a human infant).

In certain embodiments, the subject is exposed to a flavivirus due to the subject's exposure to a mosquito comprising the flavivirus. The subject may be exposed to a Aedes mosquitoes, particularly A. aegypti which live between the latitudes of 35° North and 35° South below an elevation of 1,000 metres (3,300 ft). Such a subject may be at risk of developing a flavivirus infection and disease states related to or caused by such an infection.

In certain embodiments, the subject does not have, but is at risk of developing a dengue virus infection. A subject “at risk” may or may not have detectable disease, and may or may not have displayed detectable disease prior to the treatment methods described herein. “At risk” denotes that an individual who is determined to be more likely to develop a symptom based on conventional risk assessment methods or has one or more risk factors that correlate with development of a particular condition. An individual having one or more of these risk factors has a higher probability of developing a condition than an individual without these risk factors. Examples (i.e., categories) of risk groups are well known in the art and discussed herein, such as those subjects who are traveling to a region of the world where the dengue virus is prevalent. For example, in some embodiments the region is in the United States, Argentina, Australia, Bangladesh, Barbados, Bolivia, Belize, Brazil, Cambodia, Colombia, Costa Rica, Cuba, Dominican Republic, French Polynesia, Guadeloupe, El Salvador, Grenada, Guatemala, Guyana, Haiti, Honduras, India, Indonesia, Jamaica, Laos, Malaysia, Melanesia, Mexico, Micronesia, Nicaragua, Pakistan, Panama, Paraguay, The Philippines, Puerto Rico, Samoa, Western Saudi Arabia, Singapore, Sri Lanka, Suriname, Taiwan, Thailand, Trinidad and Tobago, Venezuela, Vietnam or China.

### EXAMPLES

The invention now being generally described will be more readily understood by reference to the following examples which are included merely for purposes of illustration of certain aspects and embodiments of the present invention, and are not intended to limit the invention in any way.

### Experimental Procedures

Cell Culture and Viruses. HEK293T, Huh7, Huh7.5, Vero and A549 cells were cultured in Dulbecco's Modified Eagle's Medium (DMEM) supplemented with 10% fetal bovine serum (FBS), 10 mM HEPES and 1% penicillin-streptomycin (Gibco). BHK-21 cells were propagated in Minimum Essential Medium Alpha (MEM-a) supplemented with 10% FBS, 10 mM HEPES and 1% penicillin-streptomycin. C6/36 cells were cultured in Eagle's Minimum Essential Medium (EMEM) supplemented with 10% FBS and 1% penicillin-streptomycin, and grown at 28° C. K562 cells and primary CD14⁺ monocytes were maintained in Roswell Park Memorial Institute (RPMI) 1640 medium supplemented with 10% FBS, 1% non-essential amino acid solution (Gibco) and 1% penicillin-streptomycin. DV2 NGC, DV1 276 RK1, DV2 16681, DV3 BC188/97 and DV4 814699 were propagated in C6/36 cells. SeV (Cantell) was purchased from Charles River Laboratories. HSV-1 was a kind gift from David Knipe (Harvard).

Plasmids and Transfections pQCXIP-NS2B/3-HA and pQCXIP-NS3-HA were generated by subcloning NS2B/3 (containing NS2B and NS3) or NS3 of DV2 (strain NGC) into pQCXIP vector using NotI and BamHI sites. GST-NS3 and GST-NS5 were generated by subcloning NS3 or NS5 of DV2 (strain NGC) into pEBG vector between BamHI and Clal. Similarly, NS3 of YFV (kindly provided by Richard Kuhn, Purdue University) and NS3 of HCV (kindly provided by Zhijian Chen, UT Southwestern) were subcloned into the pEBG vector. pEF-BOS-FLAG-NS3-Pro (aa 1-179), pEF-BOS-FLAG-NS3-Hel (aa 169-618), pEF-BOS-FLAG-NS5-MTase (aa 1-319) and pEF-BOS-FLAG-NS5-Pol (aa 297-901) were generated by subcloning into pEF-BOS-FLAG vector using NotI and SalI sites. 14-3-3ε (Uniprot: P62258-1) was purchased as a cDNA clone and subcloned into pEF-BOS and pCAGGS vectors with an N-terminal FLAG and c-myc tag, respectively. HA-tagged 14-3-3σ was provided by Satoshi Inoue (University of Tokyo) and has been described (Urano et al., Nature 417, 871-875 (2002)). pQCXIP-STING-HA was generated by subcloning STING (clone ID 5762441, Thermo Scientific) into pQCXIP vector using NotI and BamHI sites. The plasmids encoding the HCV NS3/4A protease complex (pcDNA3-FLAG-NS3/4A) and its S139A catalytically-inactive mutant were a kind gift of Zhijian Chen (Li et al., PNAS 102, 17717-17722 (2005)). Plasmids encoding GST-RIG-I(2CARD), RIG-I-FLAG and TRIM25-FLAG have been described previously (Gack et al., Nature 446, 916-920 (2007); Wies et al., Immunity 38, 437-449 (2013)). The DV NS3 truncation mutants GST-NS3(1-92), GST-NS3(93-168), GST-NS3(43-92), GST-NS3(82-168), GST-NS3(63-168), and GST-NS3(43-168) were generated by PCR using GST-NS3 full-length as template. All constructs were sequenced to verify 100% agreement with the original sequence. Transfections were performed using the calcium phosphate method, or with TurboFectin 8.0 (Origene), Lipofectamine and Plus reagent, or Lipofectamine 2000 (all Life Technologies) according to the manufacturer's instructions.

14-3-3ε Knockdown Experiments. siRNAs targeting 14-3-3ε (siGENOME SMARTpool M-017302-03-0005) as well as a non-targeting control siRNA were purchased from Dharmacon. K562 cells were seeded into 12-well plates and transfected with 300 nM siRNA using Lipofectamine RNAiMAX (Life Technologies) according to the manufacturer's instructions. Knockdown of endogenous 14-3-3ε was determined by western blot analysis.

Antibodies and Reagents. For western blot analysis, the following antibodies were used: anti-FLAG (M2, Sigma), anti-HA (HA-7, Sigma), anti-GST (Sigma), anti-c-myc (9E10), anti-β-actin (Abcam), anti-RIG-I (Alme-1, Adipogen), anti-TRIM25 (BD Biosciences), anti-ubiquitin (P4D1, Santa Cruz), anti-PP1γ (Bethyl Laboratories), anti-ISG15 (F-9, Santa Cruz), anti-ISG54 (ProSci), anti-STAT2 (Santa Cruz), anti-14-3-3ε (8C3, Santa Cruz), anti-NS3 (E1D8, kindly provided by Eva Harris), anti-NS3 (GT2811, Genetex), anti-MAVS (AT107, Enzo), anti-GAPDH (CS204254, Millipore), anti-IRF3 (sc-9082, Santa Cruz). For immunoprecipitation of 14-3-3ε, anti-14-3-3ε (11648-2-AP, Proteintech) was used. For flow cytometry analysis, anti-prM (2H2, Merck Millipore) was conjugated to DyLight 633 using a commercial kit (Thermo Scientific) and used to detect DV-infected cells. Anti-CD14-FITC (M5E2, BD Biosciences) was used to determine purity of CD14⁺ monocytes. Isotype control antibodies were purchased from BD Biosciences.

Luciferase Reporter Assay. HEK293T cells were seeded into 12-well plates. The following day, cells were transfected with 200 ng IFN-β luciferase construct, 300 ng β-gal-expressing pGK-β-gal, and 100 ng-1 μg of plasmid encoding effector protein. To stimulate IFN-β promoter activity, 2 ng of GST-RIG-I-2CARD was co-transfected, or cells were infected with SeV (50 HAU/ml) 48 hours after transfection. Cells were harvested and assayed for luciferase activity (Promega). Luciferase values were normalized to β-galactosidase activity to control for transfection efficiency.

Pull-down Assay, Co-Immunoprecipitation, and Immunoblot Analysis HEK293 T or Huh7 cells were lysed in NP-40 buffer (50 mM HEPES pH 7.4, 150 nM NaCl, 1% [vol/vol] NP-40, protease inhibitor cocktail [Sigma]) and centrifuged at 13,000 rpm for 20 min. GST or FLAG pull-down, Co-IP, and western blot analyses were performed as previously described (Chan et al., PloS one 7, e34508 (2012); Gack et al., Nature 446, 916-920 (2007)).

Large-scale Protein Purification and Mass Spectrometry. HEK239T cells were transfected with pEF-BOS-FLAG-NS3-Pro, pEF-BOS-FLAG-NS3-Hel, pEF-BOS-FLAG-NS5-MTase or pEF-BOS-FLAG-NS5-Pol. Two days later, cells were lysed with NP-40 buffer supplemented with protease inhibitor cocktail (Sigma). Clarified lysates were mixed with a ˜50% slurry of anti-FLAG-conjugated sepharose beads (Sigma) and incubated for 4 h at 4° C. After extensive washing of the beads, bound proteins were eluted and separated on a NuPAGE 4-12% Bis-Tris gradient gel (Life Technologies). Coomassie staining was performed and a ˜30 kDa band specifically present in the FLAG-NS3-Pro sample was excised and analyzed by ion-trap mass spectrometry at the Harvard Taplin Biological Mass Spectrometry facility.

Confocal Microscopy. Huh7 cells were grown on chamber slides or on cover slips in 24-well plates, and then infected with DV2 or SeV at indicated titers, or mock infected. Cells were harvested at indicated time points and fixed with 4% (w/v) paraformaldehyde for 20 min, permeabilized with 0.2% (v/v) Triton-X-100 in PBS, and blocked with 10% (v/v) goat serum or FBS in PBS for 1 h. For immunostaining, anti-14-3-3ε (Proteintech), anti-NS3 (GT2811 or GTX 124252, Genetex), anti-NS4A (GTX 124249, Genetex), anti-ISG54 (12604-1-AP, Proteintech), anti-RIG-I (Alme-1, Adipogen), and anti-FLAG (Sigma, Abcam and Bethyl) were used, followed by incubation with secondary antibodies conjugated to Alexa Fluor 488, Alexa Fluor 594, or Alexa Fluor 647 (Life Technologies or Abcam). Cells were mounted in DAPI-containing Vectashield (Vector Labs) to co-stain nuclei. All laser scanning images were acquired on an Olympus IX81 confocal microscope.

Direct Protein Interaction Assay Bacterially-purified recombinant human 14-3-3ε protein (NP_006752.1) was purchased from Sino Biological. GST or GST-NS3 (DV2, strain NGC) expressed in HEK293T cells was immobilized on glutathione-conjugated sepharose beads in NP-40 buffer and incubated with recombinant 14-3-3ε protein (final concentration of 10 μg/ml) for 2 h at 4° C. After extensive washing with NP-40 buffer, bound proteins were eluted from the beads with 2× Laemmli buffer and heated at 95° C. for 5 min, followed by SDS-PAGE and western blot analysis. Similarly, TRIM25-FLAG and RIG-I-FLAG were purified from transfected HEK293T cells using anti-FLAG-conjugated sepharose beads and tested for binding to recombinant 14-3-3ε.

Mitochondria Fractionation Assay HEK293T or Huh7 cells were infected with DV or SeV at indicated titers, or mock infected. 20-24 hours later, a portion of cells was harvested for WCLs, and another portion for fractionation assay using a commercial mitochondria/cytosol fractionation kit (MIT1000, Merck Millipore) according to the manufacturer's instructions. Briefly, cells were disrupted in Isotonic Mitochondrial Buffer using a Dounce homogenizer. Lysates were subjected to low-speed centrifugation to pellet nuclei and unbroken cells. Supernatant was subsequently centrifuged at 10,000×g for 30 min at 4° C. The supernatant containing the cytosol and microsome fraction (‘cytosolic fraction’) as well as the pellet containing the enriched mitochondrial fraction were subjected to a bicinchoninic acid (BCA) assay. Equal amounts of protein were loaded for SDS-PAGE and analyzed by western blot. Anti-GAPDH and anti-MAVS western blot analyses served as controls.

Dengue Virus Infection and Flow Cytometry Analysis. Infection was performed based on a published protocol (Diamond et al., J. Virology 74, 7814-7823 (2000)). Briefly, ˜1.5×10⁵ Huh7 cells per well were seeded into 24-well plates and allowed to adhere for 4 h. Virus diluted in 250 μl DMEM containing 2% FBS was incubated at 37° C. for 1.5 h. At the indicated time points after infection, cells and/or supernatants were harvested. For K562 suspension cells, the infection was performed similarly except growth media was directly added to cells after infection. To detect DV-infected cells, cells were washed once in PBS, fixed in 1% (w/v) paraformaldehyde, permeabilized with 0.1% saponin (Sigma), and then stained with anti-prM-DyLight 633 in permeabilization buffer for ˜40 min at 4° C. Subsequently, cells were washed with PBS and resuspended in 1% (w/v) paraformaldehyde before flow cytometry analysis on a FACS Calibur (BD Biosciences). Analysis was performed using FlowJo software (Tree Star).

Bioinformatics analysis. NS3 protein sequences from full genome DV sequences were analyzed with NIAID Virus Pathogen Database and Analysis Resource (ViPR) online through the website at http://www.viprbrc.org.

Quantitative Real Time PCR (qRT-PCR). Total RNA was extracted from cells using an RNA extraction kit (OMEGA Bio-Tek). Equal amounts of RNA (typically 10-100 ng) were used in an one-step qRT-PCR reaction (SuperScript III Platinum One-Step qRT-PCR kit with ROX, Life Technologies) with commercially available primers with FAM reporter dye for the indicated target genes (IDT). Expression level for each target gene was calculated by normalizing against GAPDH using the ΔΔCT method and expressed as fold levels compared to mock-infected cells. All qRT-PCR reactions were run on a 7300 RT-PCR System or 7500 FAST RT-PCR System (both ABI).

Generation of a NS3_{KIKP} Mutant Dengue Virus. DV2_{KIKP} was generated based on an infectious clone of DV2 16681, pD2/IC-30P, kindly provided by Claire Huang (CDC) and described previously (Butrapet et al., J. Virology 74, 3011-3019 (2000); Kinney et al., Virology 230, 300-308.(1997)). PCR was used to generate mutant pD2/IC-30P harboring R64K and E66K mutations in the NS3 gene. The wild-type and mutant infectious clone plasmids were linearized by XbaI digestion and in vitro transcribed using the T7 promoter (RiboMAX Large Scale RNA Production System, Promega) with the addition of a m⁷G(5′)ppp(5′)A RNA cap structure analog (New England Biolabs). The in vitro transcribed RNA was purified using Micro Bio-Spin columns (Bio Rad) and transfected into Vero cells using Lipofectamine 2000. Viral supernatants were harvested and used to propagate the wild-type and mutant virus in Vero cells. Vero cells were further used to titer the recombinant viruses using a FACS-based assay (Lambeth et al., J. Clinical Microbiology 43, 3267-3272 (2005)) with anti-prM antibody.

DV infection studies in Primary Human Monocytes. Human peripheral blood or peripheral blood mononuclear cells (PBMCs) from unidentified healthy donors was purchased (HemaCare). In the case of human peripheral blood, PBMCs were isolated using Ficoll-Hypaque (GE Healthcare) density gradient centrifugation. CD14⁺ monocytes were positively selected from PBMCs using anti-CD14 magnetic microbeads according to the manufacturer's instructions (Miltenyi Biotec). CD14⁻ monocytes were rested overnight in growth media before use, or cryopreserved for use in future experiments. The purity of CD14⁺ cells was routinely ˜90%, as determined by anti-CD14-FITC staining (BD Biosciences) and flow cytometry analysis. For infection experiments, ˜1.5×10⁵ CD14⁺ monocytes per well were infected with DV in a 96-well plate in 250 μl DMEM containing 2% FBS for 5 h, with occasional agitation.

Statistical analysis. Unpaired two-tailed Student's t tests were used. P<0.05 was defined as statistically significant.

### Example 1

### NS3 Protein Interacts with 14-3-3ε

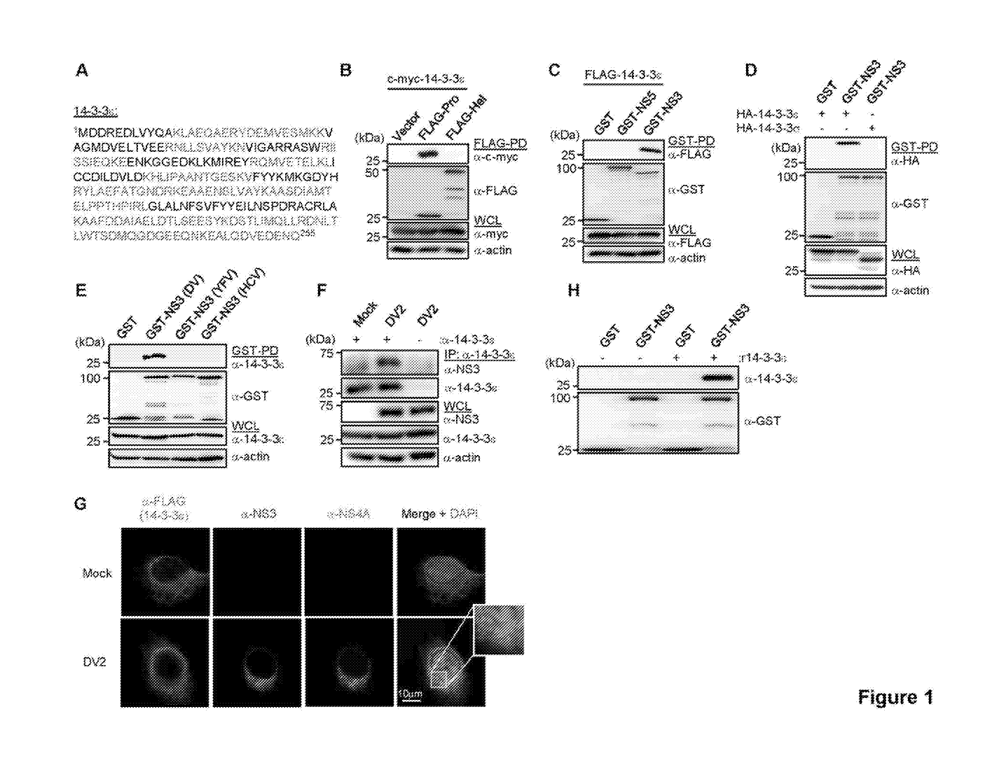

The identity of novel cellular interaction partners of NS3 and NS5 were investigated. In order to identify the interaction partners, affinity purification and mass spectrometry (MS) analysis of defined FLAG-tagged domains of both viral proteins: the NS3 protease (amino acids (aa) 1-179) and helicase (aa 169-619) domains (FLAG-NS3-Pro and FLAG-NS3-Hel), as well as the NS5 methyltransferase (aa 1-319) and polymerase (aa 297-901) domains (FLAG-NS5-MTase and FLAG-NS5-Pol) was utilized. MS analysis showed that 14-3-3ε, a ˜30 kDa mitochondrial-targeting chaperone protein, was specifically present in complex with FLAG-NS3-Pro, but not with FLAG-NS3-Hel, FLAG-NS5-MTase or FLAG-NS5-Pol (FIG. 1, Panel A and data not shown).

Using co-immunoprecipitation (Co-IP) assay, c-myc-tagged 14-3-3ε specifically bound to NS3-Pro, but not to NS3-Hel was first confirmed (FIG. 1, Panel B). Furthermore, in agreement with the MS results, ectopically expressed FLAG-14-3-3ε interacted specifically with NS3 (fused to Glutathione S-transferase; GST-NS3), but not GST-NS5 or GST alone (FIG. 1, Panel C). The interaction between NS3 and 14-3-3ε was specific as 14-3-3σ, another member of the 14-3-3 protein family which shares ˜75% homology with 14-3-3ε, did not bind GST-NS3 (FIG. 1, Panel D). Furthermore, exogenously expressed NS3 of DV strongly interacted with endogenous 14-3-3ε (FIG. 1, Panel E); in contrast, the NS3 proteins of Yellow Fever virus (YFV) and Hepatitis C virus (HCV), two related viruses that also belong to the family Flaviviridae, did not bind 14-3-3ε (FIG. 1, Panel E). Next, whether NS3 binds to 14-3-3ε during DV infection by determining the interaction of endogenous 14-3-3ε and NS3 in DV-infected human hepatoma cells (Huh7) by Co-IP was addressed. NS3 efficiently formed a complex with endogenous 14-3-3ε during DV infection (FIG. 1, Panel F). Furthermore, laser scanning confocal microscopy of DV-infected Huh7 cells showed that 14-3-3ε was expressed throughout the cytoplasm, while DV NS3, as previously reported, formed perinuclear cytoplasmic speckles, which are indicative of DV replication complexes at ER-derived membranes (Apte-Sengupta et al., Current Opinion in Virology 9C, 134-142 (2014)). NS3 co-localized extensively with 14-3-3ε in these perinuclear bodies, which also co-stained with NS4A, a key component of the DV replication complex (Miller et al., J. of Biological Chemistry 282, 8873-8882 (2007)) (FIGS. 1, Panel G). Finally, we assessed whether NS3 directly binds to 14-3-3ε by performing an in vitro binding assay (FIG. 1, Panel H). This showed that GST-NS3 immobilized on glutathione agarose beads, but not GST alone, efficiently interacted with bacterially-purified recombinant (r) 14-3-3ε, demonstrating a direct interaction between NS3 and 14-3-3ε. Taken together, these results indicate that DV NS3 and 14-3-3ε bind directly to each other and form a complex during DV infection.

### Example 2

### 14-3-3ε is Critical for Controlling DV Replication

The effect of ectopically expressed 14-3-3ε on DV replication in Huh7 cells was determined. 14-3-3ε expression suppressed DV2 replication and 14-3-3ε overexpression inhibited the replication of four other DV strains representing all four serotypes (DV1-4), but had no effect on herpes simplex virus-1 (HSV-1), an unrelated DNA virus. To determine the relevance of 14-3-3ε in restricting DV replication, 14-3-3ε expression in K562 cells was silenced using short interfering RNAs (siRNAs). Knockdown of 14-3-3ε in K562 cells significantly enhanced DV replication as compared to non-targeting control siRNA, supporting a role for 14-3-3ε in controlling DV replication.

### Example 3

### NS2B/3 Inhibits RIG-I Activation Independent of Proteolytic Activity

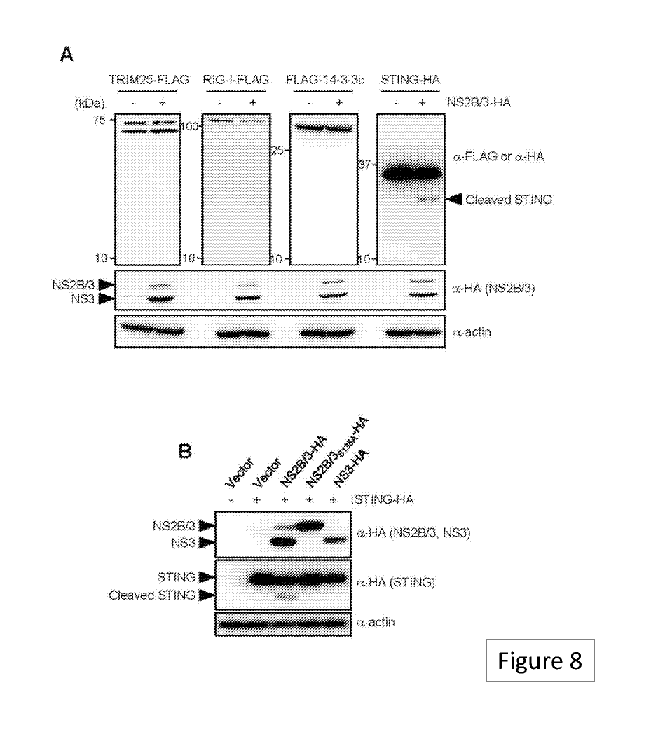

Whether NS3 in complex with NS2B can cleave 14-3-3ε was assessed. Immunoblot (IB) analysis showed that overexpression of a proteolytically-active NS2B/3 construct did not result in any cleavage products of co-expressed FLAG-14-3-3ε (FIG. 8, Panel A). In support of this, endogenous 14-3-3ε protein levels were unchanged during DV infection (FIGS. 1, Panel F and 4, Panel D), strongly suggesting that DV NS2B/3 does not cleave 14-3-3ε. Furthermore, NS2B/3 did not cleave TRIM25 or RIG-I, the two other essential components of the 14-3-3ε translocon complex (FIG. 8, Panel B). In contrast, NS2B/3 readily cleaved co-expressed STING, which served as a positive control.

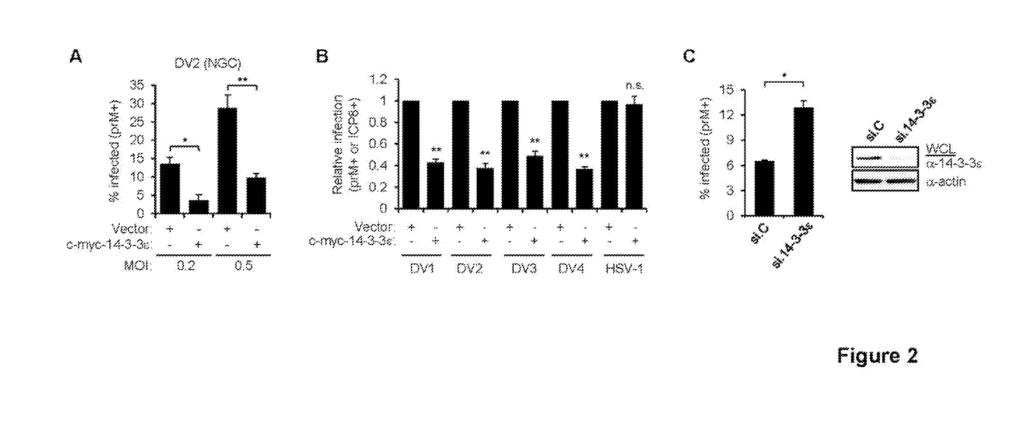

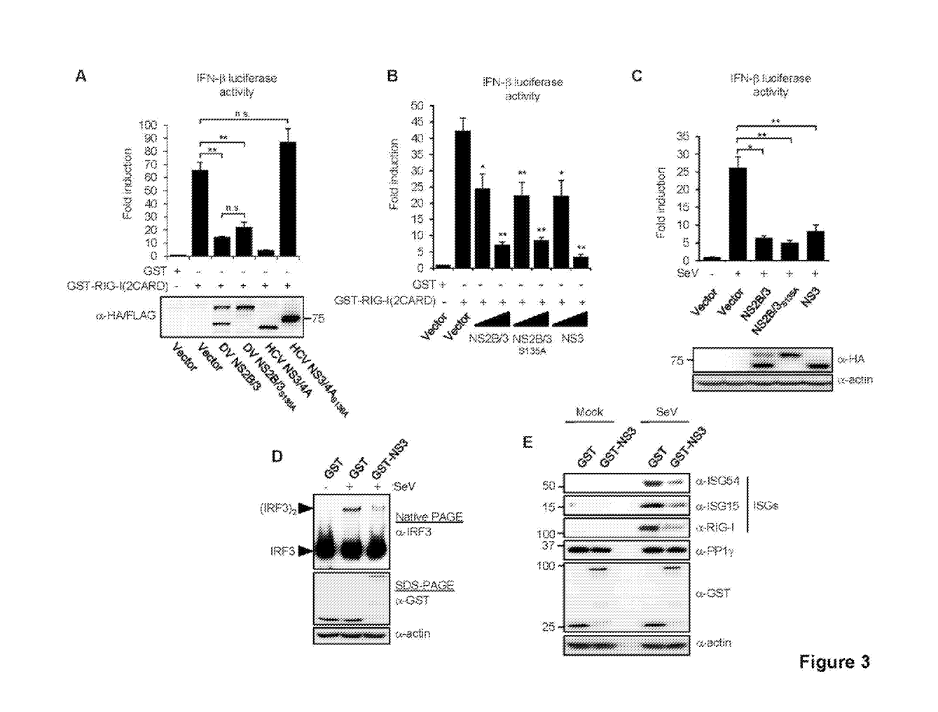

Next, whether NS3 inhibits RIG-I signaling in a cleavage-dependent or—independent manner was assessed. To address this question, a catalytically-inactive mutant of NS2B/3 (NS2B/3_{S135A}) (Khumthong et al., J. Biochem. and Mol. Bio. 35, 206-212(2002)), which, in contrast to WT NS2B/3, was unable to cleave itself or STING (Figure S2B) was generated. Ectopic expression of both NS2B/3 and NS2B/3_{S135A} potently suppressed IFN-β induction mediated by ectopic expression of RIG-I 2CARD, the constitutively active signaling module of RIG-I (FIG. 3, Panels A and B). In contrast, only WT NS3/4A of HCV suppressed RIG-I 2CARD-mediated IFN-β induction, while the inactive mutant NS3/4A_{S139A} failed to do so (FIG. 3, Panel A). NS3 alone, which has no proteolytic activity without its co-factor NS2B (Falgout et al., J. Virology 65, 2467-2475 (1991) (FIG. 8, Panel B), blocked RIG-I 2CARD- and SeV-induced IFN-β promoter activation as potently as NS2B/3 WT and NS2B/3_{S135A} (FIGS. 2B and 2C). Consistent with this finding, NS3 overexpression also inhibited endogenous IRF3 dimerization and ISG induction triggered by SeV infection (FIGS. 2D and 2E). These results indicate that DV NS3 inhibits RIG-I- and 14-3-3ε-mediated signaling in a cleavage-independent manner.

### Example 4

### NS3 Prevents Binding of the RIG-I-TRIM25 Complex to 14-3-3ε, Thereby Inhibiting the Translocation of Activated RIG-I to Mitochondria/MAMs

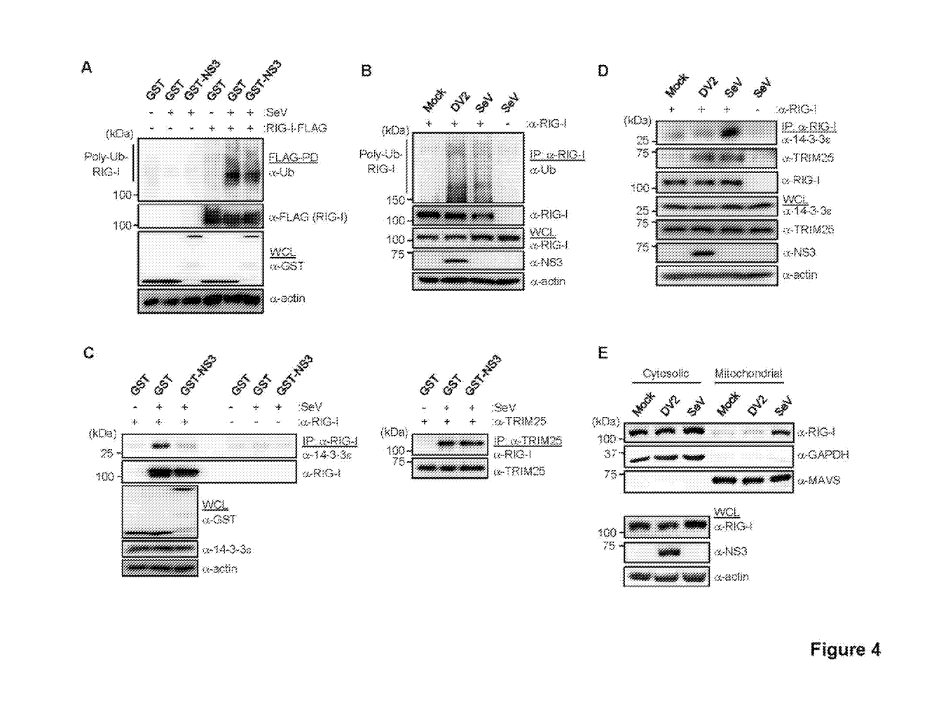

Whether NS3 (i) blocks the K63-linked ubiquitination of RIG-I, (ii) interferes with the complex formation of 14-3-3ε, RIG-I and TRIM25, or (iii) inhibits the translocation of RIG-I to mitochondria/MAMs, all of which are critical steps of RIG-I activation, was examined. Efficient ubiquitination of FLAG-RIG-I upon SeV infection was detected in both GST and GST-NS3 co-expressing cells (FIG. 4, Panel A). Robust ubiquitination of endogenous RIG-I during both DV and SeV infection was detected which indicates that DV does not inhibit the ubiquitination of RIG-I (FIG. 4, Panel B).

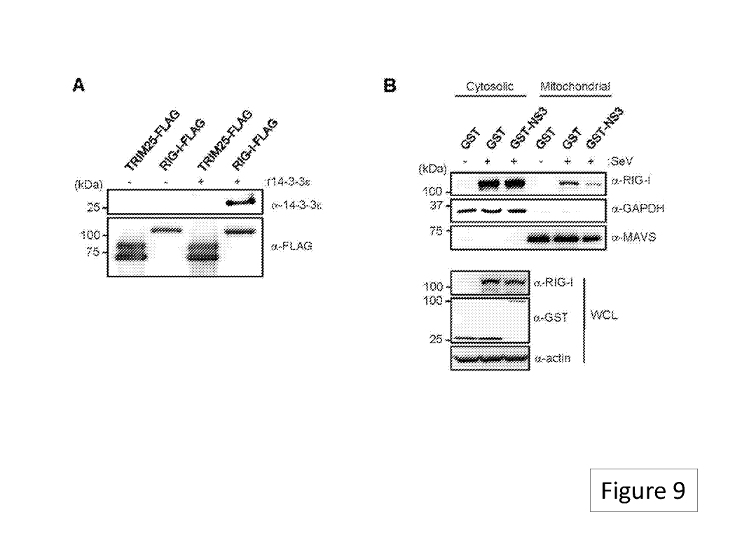

The binding of endogenous RIG-I to 14-3-3ε or TRIM25 upon SeV infection in the presence or absence of exogenous NS3 was determined. While SeV infection triggered both 14-3-3ε and TRIM25 binding to RIG-I, expression of GST-NS3, but not GST alone, reduced 14-3-3ε binding to RIG-I, but did not affect the virus-induced interaction between TRIM25 and RIG-I (FIG. 4, Panel C). In support of this, DV infection induced the complex formation of endogenous RIG-I and TRIM25 as efficiently as SeV infection (FIG. 4, Panel D). However, the interaction between 14-3-3ε and RIG-I during DV infection was minimal and comparable to the interaction observed in mock-infected cells. In contrast, SeV infection induced robust 14-3-3ε-RIG-I binding (FIG. 4, Panel D). These results indicate that NS3 does not affect TRIM25 binding and K63-linked ubiquitination of RIG-I, but instead specifically blocks the interaction of RIG-I with 14-3-3ε . In support of this, while both RIG-I and TRIM25 form a ternary complex with 14-3-3ε in infected cells (Liu et al., Cell Host & Microbe 11, 528-537 (2012)), only RIG-I-FLAG, but not TRIM25-FLAG, interacted with bacterially-purified r14-3-3ε in an in vitro binding assay (FIG. 9, Panel A).

Fractionation studies of DV- or SeV-infected Huh7 cells were performed. In mock-infected cells, RIG-I was present almost exclusively in the cytosolic fraction, whereas in SeV-infected cells, RIG-I was abundant in the mitochondrial fraction, along with MAVS, indicating its translocation from the cytosol to mitochondria/MAMs. In striking contrast, RIG-I failed to translocate to the MAVS-containing mitochondrial fraction during DV infection (FIG. 4, Panel E). The defect in RIG-I translocation was attributable to NS3 as ectopic expression of GST-NS3, but not GST alone, markedly diminished RIG-I amounts in the mitochondrial fraction of SeV-infected cells (FIG. 9, Panel B). Together, these results indicate that binding of NS3 blocks 14-3-3ε from interacting with the activated RIG-I-TRIM25 complex, thereby preventing RIG-I from translocating to mitochondria/MAMs to initiate antiviral signaling.

### Example 5

### NS3 Binds to 14-3-3ε Using a Phosphomimetic RxEP Motif

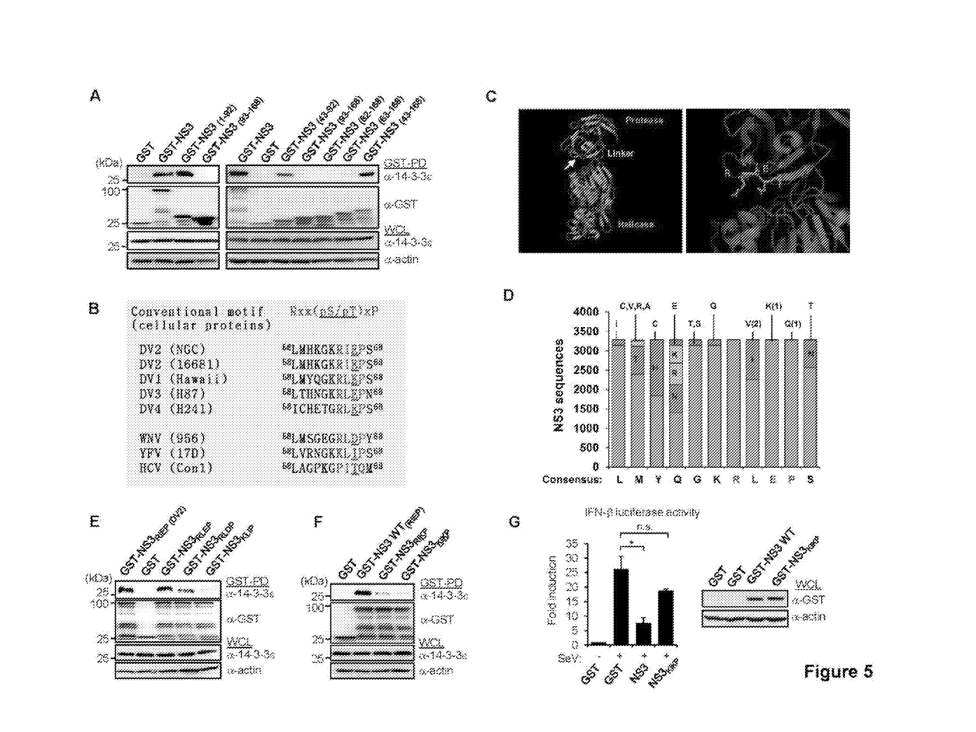

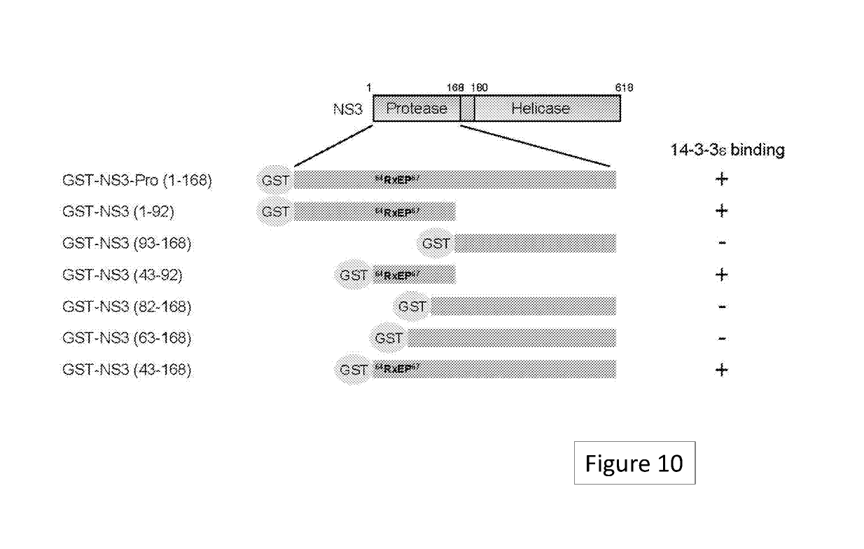

To identify the binding site of 14-3-3ε in the protease domain of NS3 (NS3-Pro), GST-fused NS3-Pro truncation fragments were constructed and tested for their abilities to bind endogenous 14-3-3ε by Co-IP. Full-length GST-NS3 served as a positive control (FIGS. 5, Panel A and FIG. 10). Full-length GST-NS3, GST-NS3 ₁₋₉₂ and GST-NS3₄₃₋₉₂ efficiently interacted with endogenous 14-3-3ε, while other NS3 fragments did not bind 14-3-3ε under the same conditions.

A hallmark of many cellular proteins that bind to 14-3-3 family members is the presence of a canonical high-affinity binding motif, such as Rxx(pS/pT)xP, where x denotes any residue and pS/pT indicates a phosphorylated serine/threonine residue (Mhawech, Cell Research 15, 228-236 (2005)). Phosphorylation of S/T in Rxx(pS/pT)xP has been shown to be essential for 14-3-3 binding, as dephosphorylation of this residue abrogates 14-3-3 interaction (Yaffe et al., Cell 91, 961-971 (1997)). A closer examination of NS₄₃₋₉₂, which is sufficient for 14-3-3 binding (FIG. 5, Panel A), revealed a “compact” ⁴⁶RIEP⁶⁷ motif bearing close similarity to the cellular Rxx(pS/pT)xP motif but harboring a charged Glu⁶⁶ (E⁶⁶) residue in place of pS/pT (FIG. 5, Panel B). Based on the crystal structure of the DV NS3 protein (Luo et al., J. Virology 82, 173-183 (2008)), which revealed an elongated shape of NS3 in which the linker region between the Pro and Hel domains adopts an extended conformation, this ⁶⁴RIEP⁶⁷ motif is likely exposed for 14-3-3ε binding without steric hindrance (FIG. 5, Panel C). Furthermore, the ⁶⁴RIEP⁶⁷ motif is conserved between the DV2 strains NGC and 16681, while DV1, 3 and 4 harbor a similar motif, ⁶⁴RLEP⁶⁷ (FIG. 5, Panel B), suggesting that the ⁶⁴RxEP⁶⁷ motif is conserved across various DV strains. Indeed, bioinformatics analysis aligning more than 3000 NS3 sequences derived from fully-sequenced DV1-4 strains showed that the ⁶⁴RxEP⁶⁷ motif is conserved in all except two analyzed NS3 sequences, while adjacent residues show substantially more polymorphisms (FIG. 5, Panel D).

To assess whether DV NS3 utilizes the phosphomimetic E⁶⁶ residue in ⁶⁴RxEP⁶⁷ for 14-3-3ε binding, the corresponding motif from DV1, 3 and 4 (RLEP), or WNV (RLDP), both harboring phosphomimetic residues at position 66 (E⁶⁶ or D⁶⁶), were transplanted into full-length NS3 derived from DV2 (NGC strain, which contains RIEP). In addition, the corresponding motif from YFV (KLIP), harboring an uncharged hydrophobic residue at position 66 (1⁶⁶), were transplanted into DV2 NS3. Chimeric NS3 proteins containing RLEP and RLDP (NS3_{RLEP} and NS3_{RLDP}), which harbor E⁶⁶ or D⁶⁶, were both able to bind 14-3-3ε. In contrast, NS3_{KLIP} showed strongly diminished binding (FIG. 5, Panel E), which is consistent with our results that YFV NS3 does not bind 14-3-3ε (FIG. 1, Panel E). This suggests that D⁶⁶, which is also a phosphomimetic residue, can substitute for E⁶⁶, and that I⁶⁵ and L⁶⁵ are functionally equivalent.

To further probe the importance of the phosphomimetic E⁶⁶ residue in NS3 for 14-3-3ε binding, E⁶⁶ was replaced with Lys (K⁶⁶), a positively charged amino acid (NS3_{RIKP}). NS3_{RIKP} exhibited profoundly diminished binding to 14-3-3ε, indicating that the phosphomimetic E⁶⁶ (or D⁶⁶) is critical for 14-3-3ε interaction. Furthermore, additional mutation of R⁶⁴ to K⁶⁴ (NS3_{KIKP}) led to a near-complete loss of 14-3-3ε binding, demonstrating the importance of E⁶⁶ and R⁶⁴ for NS3 interaction with 14-3-3ε (FIG. 5, Panel F). Collectively, these results indicate that instead of utilizing a canonical pS/pT-containing 14-3-3-binding motif, DV NS3 binds 14-3-3ε using a compact RxEP motif that contains a phosphomimetic (E⁶⁶) residue.

### Example 6

### The 14-3-3ε-Binding Deficient NS3_{KIKP} Mutant Protein is Impaired in Suppression of RIG-I Translocation and IFN-β Induction

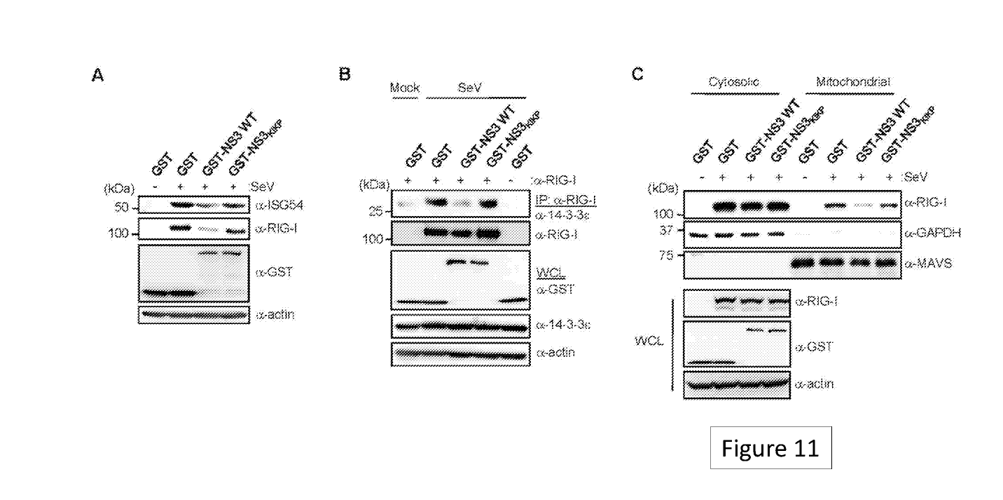

The NS3_{KIKP} mutant protein that exhibited a near-complete loss of 14-3-3ε binding was characterized functionally. The inhibitory effect of WT NS3 and the NS3_{KIKP} mutant on the complex formation of endogenous RIG-I and 14-3-3ε triggered by SeV infection were compared. While WT NS3 potently inhibited SeV-induced RIG-I-14-3-3ε binding, NS3_{KIKP} did not affect their interaction (FIG. 11, Panel A). In line with this, while WT NS3 potently blocked the translocation of endogenous RIG-I to MAVS-containing mitochondrial fractions, NS3_{KIKP} expression did not inhibit RIG-I translocation (FIG. 5, Panel G). Furthermore, expression of WT NS3, but not NS3_{KIKP}, significantly suppressed SeV-mediated IFN-β transcriptional activation and ISG protein expressions (FIG. 11, Panels B and C). These data show that a mutant NS3 protein that is deficient in 14-3-3ε binding is unable to inhibit RIG-I-14-3-3ε binding and RIG-I translocation to mitochondria/MAMs, and thus has an impaired ability to suppress IFN and ISG induction. Together, this demonstrates that NS3′s capacity to block the RIG-I-14-3-3ε interaction and RIG-I translocation is contingent on its ability to bind 14-3-3ε.

### Example 7

### A Recombinant DV Encoding a NS3_{KIKP} Mutant Protein is Attenuated in Replication and Elicits Enhanced Levels of IFNs, ISGs and Proinflammatory Cytokines

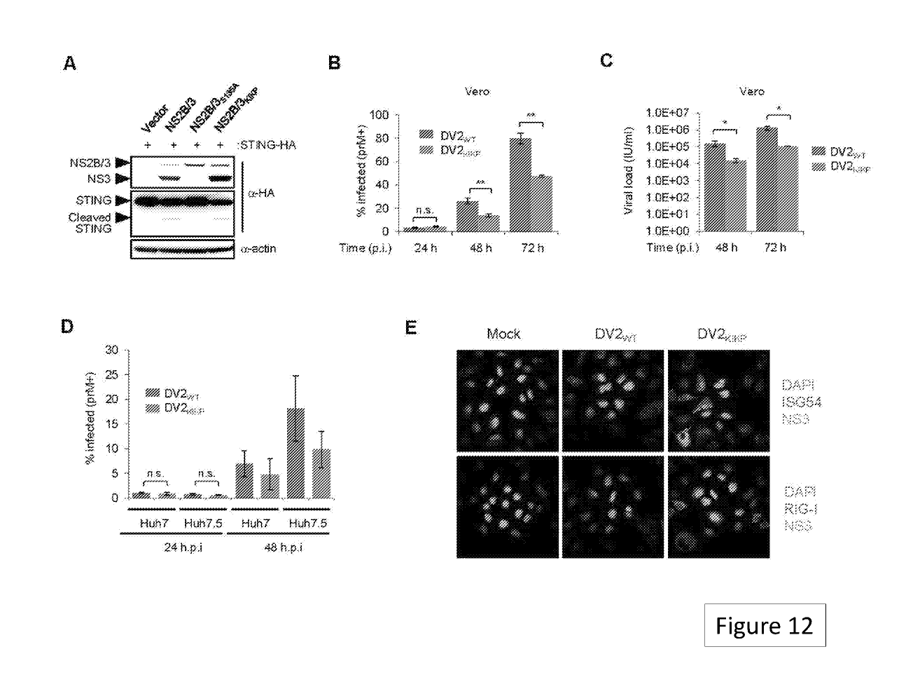

A recombinant DV encoding the NS3_{KIKP} mutant protein that is impaired in 14-3-3ε binding and RIG-I antagonism was constructed. Since NS3, as part of the NS2B/3 protease complex, processes the viral polyprotein and is therefore essential for DV replication, whether a NS2B/3_{KIKP} mutant protein retains proteolytic activity was determined. IB analysis showed that similar to WT NS2B/3, NS2B/3_{KIKP} was able to induce self-cleavage (FIG. 12, Panel A), which confirms an intact proteolytic activity and further suggests that a recombinant DV encoding NS2B/3_{KIKP} would be viable. Using reverse genetics, the R⁶⁴→K⁶⁴ and E⁶⁶→K⁶⁶ mutations in an infectious clone originally derived from DV2 16681 (Butrapet et al., J. Virology 74, 3011-3019 (2000); Kinney et al., Virology 230, 300-308 (1997)) were introduced, thereby engineering a recombinant mutant DV encoding NS3_{KIKP} (subsequently referred to as DV2_{KIKP}).

Assessment of virus replication in Vero cells, which are deficient in type I IFN responses (Desmyter et al., J. Virology 2, 955-961 (1968)), showed that DV2_{KIKP} exhibited reduced replication capacity compared to the parental virus (DV2_{WT}), resulting in approximately 1-log lower viral loads of DV2_{KIKP} compared to DV2_{WT} at 48 h and 72 h postinfection (FIG. 12, Panels B and C). A decreased replication efficiency of DV2_{KIKP}, compared to WT virus, was also observed in mosquito (C6/36) and hamster (BHK21) cells (data not shown), indicating that the slightly attenuated replication capacity of DV2_{KIKP} is independent of the host type I IFN system.

Next, the replication of DV2_{WT} and DV2_{KIKP} we tested in Huh7 cells, which have an intact type I IFN response. The replication rates of both DV2_{WT} and DV2_{KIKP} were similar 24 h after infection (FIG. 12, Panel D); however, at 72 h postinfection, the replication of DV2_{KIKP} was strongly suppressed, while DV2_{WT} replicated efficiently (FIG. 5, Panel A). To determine if the strongly diminished replication of DV2_{KIKP} is due to its inability to antagonize RIG-I, the replication of DV2_{KIKP} was determined in Huh7.5 cells, a sub-cell line of Huh7 naturally harboring a RIG-I mutant protein (RIG-I T55I) that is defective in TRIM25 binding and thus ubiquitination-mediated RIG-I activation (Gack et al., PNAS 105, 16743-16748 (2008); Sumpter et al., J. Virology 79, 2689-2699 (2005))DV2_{KIKP} replicated almost as efficiently as DV2_{WT} in Huh7.5 cells (FIG. 6, Panel A), indicating that the failure of DV2_{KIKP} to replicate in Huh7 cells is predominantly due to RIG-I activation. Additionally, DV2_{WT} showed only a marginal increase in replication in Huh7.5 cells as compared to Huh7 cells (FIG. 6, Panel A), suggesting that RIG-I signaling is effectively antagonized by DV2_{WT}.

To assess whether the reduced replication capacity of DV2_{KIKP} in Huh7 cells, as compared to DV2_{WT}, is due to its inability to block IFN induction, the gene upregulation of IFN-β, ISGs, and proinflammatory cytokines upon infection with DV2_{KIKP} or DV2_{WT} was determined. To account for the differences in replication efficiency, Huh7 cells were infected with DV2_{WT} or DV2_{KIKP} using MOIs (MOI 0.3 and 1, respectively) that resulted in comparable infectivity (˜75% of cells infected at 2 d postinfection as determined by flow cytometry [data not shown]). DV2_{KIKP} elicited markedly higher levels of IFNB1, ISGs (ISG15, IFIH1 and MX1), and proinflammatory cytokines (TNF, IL6 and CCL5) than DV2_{WT} (FIG. 6, Panel B). In line with this, DV2_{KIKP} infection of A549 cells robustly induced ISG protein expression (ISG54 and RIG-I) in neighboring non-infected cells as determined by confocal immunofluorescence microscopy. In contrast, ISG protein induction was low in response to DV2_{WT} infection (FIG. 12, Panel E). Crucially, we found that the higher IFNB1 induction by DV2_{KIKP}, as compared to DV2_{WT}, was mediated by RIG-I activation, as Huh7.5 cells that are defective in RIG-I signaling exhibited low IFNB1 induction following DV2_{KIKP} infection (FIG. 6, Panel C). To rule out the possibility that the impaired capacity of DV2_{KIKP} to inhibit ISG induction was due to a potential defect in its ability to degrade STAT2, the degradation of endogenous STAT2 in infected Huh7 cells was tested. Both DV2_{WT} and DV2_{KIKP} were able to effectively degrade STAT2. Furthermore, a loss of STING antagonism could not account for the higher IFN response induced by DV2_{KIKP}, as both WT NS2B/3 and NS2B/3_{KIKP} efficiently cleaved STING (FIG. 12, Panel A). Mechanistically, cells infected with DV2_{KIKP} exhibited efficient RIG-I translocation to the mitochondrial fraction. In contrast, DV2_{WT} infection marginally induced RIG-I translocation (FIG. 6, Panel D). Taken together, these results indicate that the 14-3-3ε-binding-deficient DV2_{KIKP} virus fails to antagonize RIG-I and thus elicits enhanced induction of IFNs, ISGs and proinflammatory cytokines. This augmented immune response in turn contributes to restriction of DV2_{KIKP} replication.

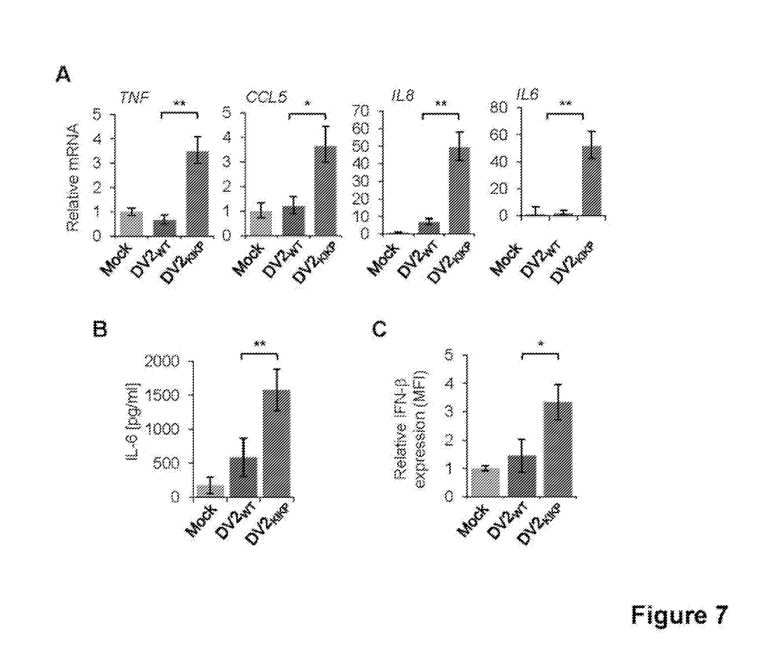

While the liver is commonly involved during DV infection in vivo, mononuclear phagocytes are thought to be the primary in vivo cell targets for DV replication (Jessie et al., The J. Infectious Diseases 189, 1411-1418 (2004)). Therefore, primary human CD14⁺ monocytes with DV2_{WT} or DV2_{KIKP} (both at an MOI of 1) were infected and then measured IFNB1 induction 24 h postinfection by qRT-PCR. IFNB1 induction by both DV2_{WT} and DV2_{KIKP} was below the detection limit (data not shown), which in agreement with previous studies is likely due to the low infectivity of primary monocytes in vitro (Kou et al., Virology 410, 240-247 (2011)). However, when the gene expression of the proinflammatory cytokines TNF, CCL5, IL8 and IL6, all of which are strongly induced in monocytes upon viral infection, was measured, a robust induction of these cytokines by DV2_{KIKP}, but not DV2_{WT} (FIG. 7, Panel A) was detected. Consistent with this, CD14⁻ monocytes infected with DV_{KIKP} exhibited enhanced IL-6 protein secretion as compared to cells infected with DV2_{WT} (FIG. 7, Panel B). Collectively, these results show that a mutant DV encoding a 14-3-3ε-binding-deficient NS3 protein is impaired in its ability to antagonize RIG-I and thus elicits an augmented innate immune response in human hepatocytes and primary human monocytes.

### Example 8

### The Dengue DV2_{KIKP} Mutant Virus Elicits an Augmented T Cell Response

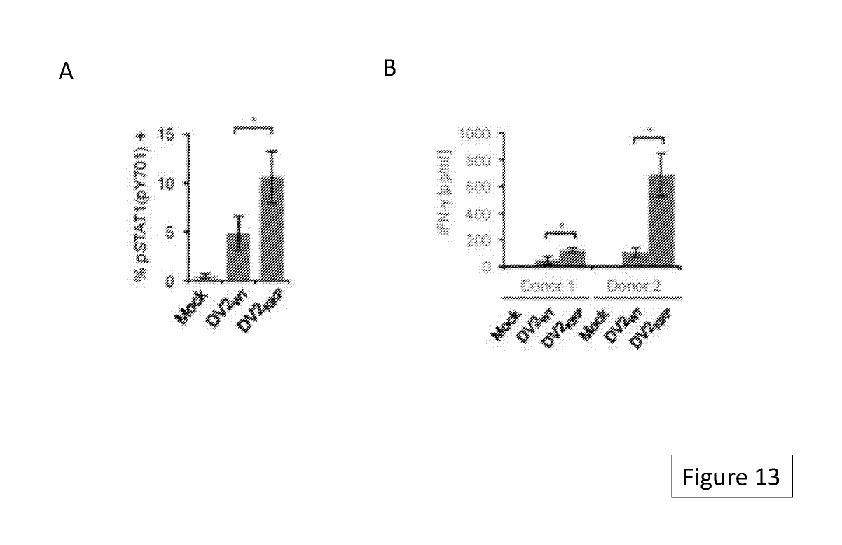

To test if the generated mutant dengue virus (DV2_{KIKP}) differentially affects the adaptive immune response compared to WT dengue virus (DV2_{WT}), primary monocyte-derived dendritic cells (moDCs) were infected with DV2_{WT} or DV2_{KIKP} and then co-cultured with syngeneic naïve pan T cells. 72 hours after infection, T cells co-cultured with DV2_{KIKP}-infected moDCs showed increased STAT1 phosphorylation (pSTAT1) when compared to DV2_{WT}, indicative of augmented interferon-α/β receptor (IFNAR) signaling (FIG. 13, Panel A). Furthermore, T cells co-cultured with DV2_{KIKP}-infected moDCs secreted significantly higher levels of IFN-at 96 hours after infection as compared to T cells co-cultured with DV2WT-infected moDCs, demonstrating greater T cell activation by DV2_{KIKP} (FIG. 13, Panel B). Taken together, these results suggest that DV2_{KIKP}, which elicits an augmented innate immune response, also elicits a stronger adaptive immune response in primary cells.

### Example 9

### West Nile Virus (WNV) NS3 Protein Antagonizes RIG-I Activation

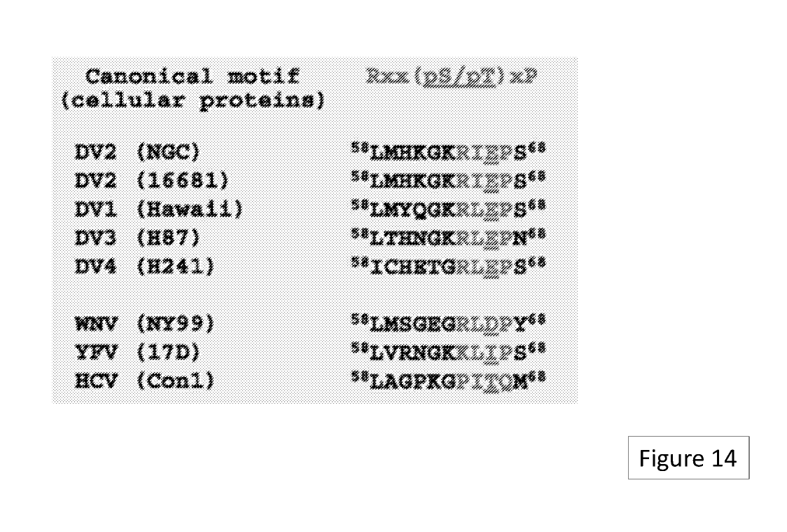

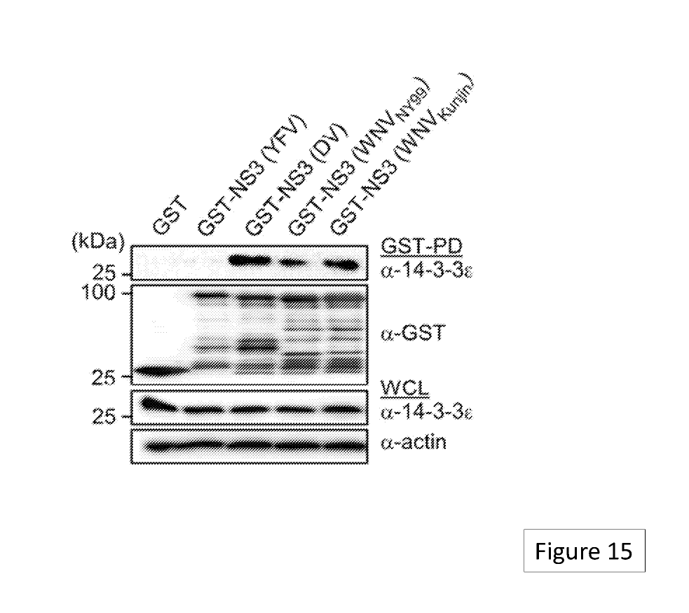

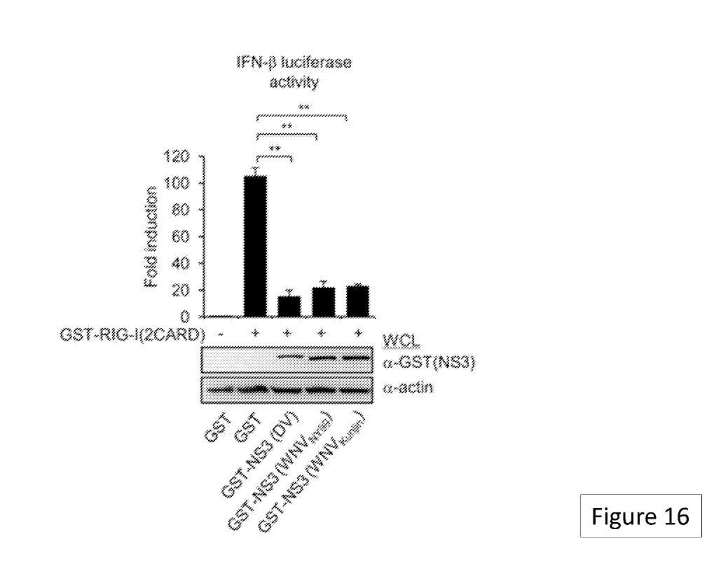

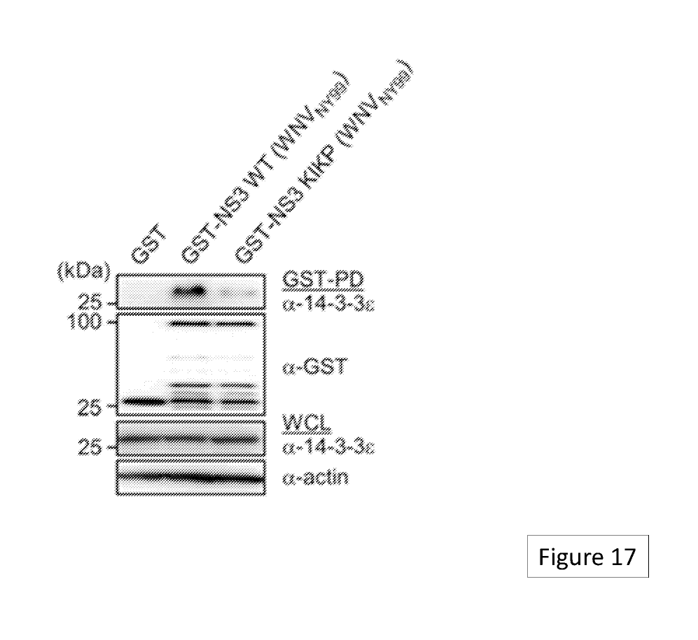

As discussed herein, dengue virus NS3 harbors a RxEP motif to usurp 14-3-3ε binding. A sequence alignment of multiple flavivirus NS3 proteins shows that West Nile virus harbors a RLDP motif (FIG. 14). As D is also a phosphomimetic residue like E, it was determined whether WNV NS3 binds 14-3-3ε to antagonize RIG-I activation and type-I interferon (IFN) induction. NS3 of both NY99 and Kunjin strains of WNV were able to bind endogenous 14-3-3ε (FIG. 15). In agreement, ectopic expression of both WNV NS3 proteins suppressed RIG-I(2CARD)-mediated IFN-induction (FIG. 16), demonstrating that WNV NS3 also blocks RIG-I signaling. Finally, mutation of the RLDP motif in WNV NS3 (NY99 strain) to KIKP strongly diminished 14-3-3ε binding, similar to results disclosed with dengue virus NS3 (FIG. 17). Taken together, this indicates that WNV uses a phosphomimetic motif to target 14-3-3 and RIG-I.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

What is claimed is:

1. A mutant flavivirus comprising a mutated NS3 protein, wherein the mutated NS3 protein is deficient in 14-3-3ε binding.

2. The mutant virus of claim 1, wherein the flavivirus is dengue virus, West Nile virus, or Zika virus.

3. The mutant virus of claim 1 or 2, wherein the mutated NS3 protein comprises a mutation between amino acid 63 and amino acid 67 relative to the amino acid sequence of SEQ ID NO: 1, SEQ ID NO: 2, or SEQ ID NO:3.

4. The mutant virus of any one of claims 1-3, wherein the mutation is an amino acid substitution, insertion, deletion, or combination thereof.

5. The mutant virus of any one of claims 1-4, wherein the mutation comprises a substitution of at least one amino acid between amino acid 63 and amino acid 67 relative to the amino acid sequence of SEQ ID NO: 1, SEQ ID NO: 2, or SEQ ID NO:3 with a different amino acid.

6. The mutant virus of any one of claims 1-4, wherein the mutation comprises substitution of at least one amino acid between amino acid 63 and amino acid 67 relative to the amino acid sequence of SEQ ID NO: 1, SEQ ID NO: 2, or SEQ ID NO:3 with lysine.

7. The mutant virus of any one of claims 1-4, wherein the mutation comprises a substitution of the amino acid at position 66 relative to the amino acid sequence of SEQ ID NO: 1, SEQ ID NO: 2, or SEQ ID NO:3 with lysine.

8. The mutant virus of any one of claims 1-4, wherein the mutation comprises a substitution of the amino acid at position 64 relative to the amino acid sequence of SEQ ID NO: 1, SEQ ID NO: 2, or SEQ ID NO:3 with lysine.

9. The mutant virus of any one of claims 1-4, wherein the mutation comprises a substitution of the amino acids at positions 64 through 66 relative to the amino acid sequence of SEQ ID NO: 1, SEQ ID NO: 2, or SEQ ID NO:3 with the amino acid sequence lysine-isoleucine-lysine.

10. The mutant virus of any one of claims 2-9, wherein flavivirus is a dengue virus serotype 1, a dengue virus serotype 2, a dengue virus serotype 3, or a dengue virus serotype 4.

11. The mutant virus of claim 10, wherein the attenuated dengue virus is a dengue virus serotype 2.

12. A pharmaceutical composition comprising the mutant virus of any preceding claim, and a pharmaceutically acceptable carrier.

13. A flavivirus vaccine comprising the mutant virus of any one of claims 1-11.

14. The vaccine of claim 13, wherein the mutant virus is selected from mutant dengue virus, mutant West Nile virus, and mutant Zika virus

15. The vaccine of claim 13 or 14, further comprising an adjuvant.

16. The vaccine of claim of any one of claims 13-15, wherein the flavivirus is a live virus.

17. A mutant NS3 protein comprising a mutation between amino acid 63 and amino acid 67 relative to the amino acid sequence of SEQ ID NO: 1 SEQ ID NO: 2, or SEQ ID NO:3 .

18. The mutant NS3 protein of claim 17, wherein the mutation comprises an amino acid substitution, insertion, deletion, or combination thereof.

19. The mutant NS3 protein of claim 17 or 18, wherein the mutation comprises a substitution of at least one amino acid between amino acid 63 and amino acid 67 relative to the amino acid sequence of SEQ ID NO: 1, SEQ ID NO: 2, or SEQ ID NO:3 with a different amino acid.

20. The mutant NS3 protein of any one of claims 17-19, wherein the mutation comprises a substitution of at least one amino acid between amino acid 63 and amino acid 67 relative to the amino acid sequence of SEQ ID NO: 1, SEQ ID NO: 2, or SEQ ID NO:3 with lysine.

21. The mutant NS3 protein of any one of claims 17-20, wherein the mutation comprises a substitution of the amino acid at position 66 relative to the amino acid sequence of SEQ ID NO: 1, SEQ ID NO: 2, or SEQ ID NO:3 with lysine.

22. The mutant NS3 protein of any one of claims 17-20, wherein the mutation comprises a substitution of the amino acid at position 64 relative to the amino acid sequence of SEQ ID NO: 1, SEQ ID NO: 2, or SEQ ID NO:3 with lysine.

23. The mutant NS3 protein of any one of claims 17-20, wherein the mutation corresponds to substituting the amino acids at positions 64 through 66 relative to the amino acid sequence of SEQ ID NO: 1, SEQ ID NO: 2, or SEQ ID NO:3 with the amino acid sequence lysine-isoleucine-lysine.

24. A virus comprising the mutant NS3 protein according to any one of claims 17-23.

25. A nucleic acid encoding the mutant NS3 protein according to any one of claims 17-23.

26. A virus comprising the nucleic acid of claim 25.

27. An expression vector comprising the nucleic acid of claim 25.

28. A cell comprising the nucleic acid of claim 25 or the expression vector of claim 27.

29. A method for producing a mutant NS3 protein, the method comprising culturing the cell of claim 28 under conditions suitable for protein expression to thereby produce the mutant NS3 protein.

30. The method of claim 29, further comprising isolating the mutant NS3 protein

31. A method for inducing in a subject an immune response against a flavivirus comprising administering to the subject a composition comprising the mutant virus of any one of claims 1-11.

32. The method of claim 31, wherein the subject is a human.

33. The method of claim 31 or 32, wherein the subject had been exposed to a flavivirus .

34. The method of claim 33, wherein the subject was exposed to dengue virus within the last 6 month, within the last month, within the last two weeks, within the last week, within the last 72 hours, within the last 48 hours, within the last 24 hours, within the last 12 hours, within the last 6 hours, within the last 4 hours, within the last 2 hours, or within the last hour.

35. The method of any one of claims 31-34, wherein the subject had been exposed to a mosquito comprising the dengue virus.

36. The method of claim 35, wherein the subject was exposed to the mosquito within the last 6 months, within the last month, within the last two weeks, within the last week, within the last 72 hours, within the last 48 hours, within the last 24 hours, within the last 12 hours, within the last 6 hours, within the last 4 hours, within the last 2 hours, or within the last hour.

37. The method of any one of claims 31-36, wherein the subject is at risk of developing a flavivirus infection.

38. The method of any one of claims 31-37, wherein the subject has a flavivirus infection.

39. The method of any one of claims 31-39, wherein the subject is traveling to a region where a flavivirus is prevalent.

40. The method of claim 40, wherein the region is in the United States, Argentina, Australia, Bangladesh, Barbados, Bolivia, Belize, Brazil, Cambodia, Colombia, Costa Rica, Cuba, Dominican Republic, French Polynesia, Guadeloupe, El Salvador, Grenada, Guatemala, Guyana, Haiti, Honduras, India, Indonesia, Jamaica, Laos, Malaysia, Melanesia, Mexico, Micronesia, Nicaragua, Pakistan, Panama, Paraguay, the Philippines, Puerto Rico, Samoa, Western Saudi Arabia, Singapore, Sri Lanka, Suriname, Taiwan, Thailand, Trinidad and Tobago, Venezuela Vietnam and/or China.

41. A method for protecting a subject from a flavivirus, comprising administering to the subject the mutant virus of any one of claims 1-11.

42. The method of claims 42, wherein the subject is a human.

43. The method of claim 41 or 42, wherein the subject is exposed to a flavivirus.

44. The method of any one of claims 41-43, wherein the subject is exposed to a mosquito comprising a flavivirus.

45. The method of any one of claims 41-44, wherein the subject does not have, but is at risk of developing aflavivirus infection.

46. The method of any one of claims 41-45, wherein the subject is traveling to a region where a flavivirus is prevalent.

47. The method of claim 46, wherein the region is in the United States, Argentina, Australia, Bangladesh, Barbados, Bolivia, Belize, Brazil, Cambodia, Colombia, Costa Rica, Cuba, Dominican Republic, French Polynesia, Guadeloupe, El Salvador, Grenada, Guatemala, Guyana, Haiti, Honduras, India, Indonesia, Jamaica, Laos, Malaysia, Melanesia, Mexico, Micronesia, Nicaragua, Pakistan, Panama, Paraguay, the Philippines, Puerto Rico, Samoa, Western Saudi Arabia, Singapore, Sri Lanka, Suriname, Taiwan, Thailand, Trinidad and Tobago, Venezuela Vietnam or China.

48. The method of any one of claims 41-47, wherein the flavivirus is dengue virus, West Nile virus, or Zika virus.

49. A method of treating a viral infection, the method comprising administering the mutant virus of any one of claims 1-11, the mutant NS3 protein of any one of claims 17-23, the pharmaceutical composition of claim 12, the vaccine of any one of claims 13-16 , the virus of claim 24 or 26, or combinations thereof to a subject.
